(19) United States
(12) Patent Application Publication
(10) Pub. No.: US 2019/0175339 A1
(43) Pub. Date: Jun. 13, 2019

(54) SEPTOMARGINAL TRABECULA ATTACHMENT FOR HEART VALVE REPAIR

(71) Applicant: VDYNE, LLC, DALLAS, TX (US)

(72) Inventor: ROBERT M. VIDLUND, FOREST LAKE, MN (US)

(21) Appl. No.: 15/838,374

(22) Filed: Dec. 12, 2017

(51) Int. Cl. A61F 2/24

(52) U.S. Cl. CPC A61F 2/2409; A61F 2/2418; A61F 2210/0014; A61F 2230/0065; A61F 2220/0083; A61F 2230/001; A61F 2220/0016

(57) ABSTRACT

The invention relates to a medical prosthesis, and in particular a tether platform for attaching to or mounting on the septomarginal trabecula of the right ventricle for securing and positioning heart valve repair devices, and in particular for securing a heart valve substitute comprising a pliant tubular conduit mounted on a resilient annular frame and tethered to a non-perforating anchor within the right ventricle of the heart, wherein the pliant tubular conduit is a reciprocating mechanical member that is compressed by pressurized working fluid within the ventricle during systole.

### CROSS-REFERENCE TO RELATED APPLICATIONS

Provided per USPTO rules by Application Data Sheet.

### STATEMENT REGARDING FEDERALLY SPONSORED R&D

Provided per USPTO rules by Application Data Sheet.

### NAMES OF PARTIES TO JOINT RESEARCH AGREEMENT

Provided per USPTO rules by Application Data Sheet.

### REFERENCE TO SEQUENCE LISTING

Provided per USPTO rules by Application Data Sheet.

### STATEMENT RE PRIOR DISCLOSURES

Provided per USPTO rules by Application Data Sheet.

### BACKGROUND OF THE INVENTION

### Field of the Invention

The invention relates to a medical prosthesis (Class 623), and in particular an attachment or mounting device in the right ventricle for securing and positioning heart valve repair devices.

### DESCRIPTION OF THE RELATED ART

In 1952 surgeons implanted the first mechanical heart valve. This first valve was a ball valve and it was designed by Dr. Charles Hufnagel. The recipient of this valve was a 30-year-old woman who could lead a normal life after the surgery. However, one downside of this design was that it could only be placed in the descending aorta instead of the heart itself. For this reason it did not fully correct the valve problem, only alleviate the symptoms. However it was a significant achievement because it proved that synthetic materials could be used to create heart valves.

In 1960, a new type of valve was invented and was successfully implanted. This valve is the Starr-Edwards ball valve, named after its originators. This valve was a modification of Hufnagel's original valve. The ball of the valve was slightly smaller and caged from both sides so it could be inserted into the heart itself.

The next development was tilting disc technology which was introduced in the late 1960s. These valves were a great improvement over the ball designs. The tilting dic technology allowed blood to flow in a more natural way while reducing damage to blood cells from mechanical forces. However, the struts of these valves tended to fracture from fatigue over time. As of 2003, more than 100,000 Omniscience and 300,000 Hall-Kaster/Medtronic-Hall tilting disc valves were implanted with essentially no mechanical failure.

In 1977, bi-leaflet heart valves were introduced by St. Jude. Similar to a native heart valve, blood flows directly through the center of the annulus of pyrolytic carbon valves mounted within nickel-titanium housing which makes these valves superior to other designs. However, a downside of this design is that it allows some regurgitation. A vast majority of mechanical heart valves used today have this design. As of 2003, more than 1.3 million St. Jude valves were deployed and over 500,000 Carbomedics valves with no failures to leaflets or housing. It should be noted that the human heart beats about 31 million times per year.

Development continues with compressible valves that are delivered via a catheter instead of requiring the trauma and complications of open heart surgery. This means that a cardiologist trained in endoscopy can, in theory, deploy a heart valve replacement during an outpatient procedure. However, transcatheter valves are often delivered by perforating the apex of the heart to access the ventricle, and the perforation is often used to anchor an annular valve replacement. Additionally, stent-style replacement valves often continue to have the regurgitation or leakage problems of prior generations of valves, as well as require expensive materials engineering in order to cope with the 100's of millions of cycles encountered during just a few years of normal heart function. Accordingly, there is still a need for alternative and simpler solutions to addressing valve-related heart pathologies.

### BRIEF SUMMARY OF THE INVENTION

Accordingly, the present invention is directed to a medical implant, comprising: a tether platform for attaching one or more tethers to the septomarginal trabecula (moderator band) of the right ventricle for securing and positioning a heart valve repair device, said tether platform sized for transcatheter delivery and deployment within the right ventricle; said tether platform having a moderator band tissue anchor and an attachment head; said moderator band tissue anchor selected from a belt anchor, a piercing anchor, and a combination thereof; said belt anchor comprising a strip or loop of material configured to encircle the septomarginal trabecula, said belt anchor having a structure selected from a mesh, a fabric, a braid, a coil, windings, and combinations thereof, said belt anchor constructed of material selected from a biocompatible tissue, a Nitinol wire, a Nitinol mesh, a polymer, and combinations thereof; said belt anchor connected to an attachment head comprising one or more tether stays, said tether stays selected from a loop, a cleat, an anchor, a fastener, a linkage, and combinations thereof; said piercing anchor comprising a structurally rigid material configured to penetrate tissue of the septomarginal trabecula, said piercing anchor having a structure selected from a helical tissue anchor, a screw-type tissue anchor, an accordion-style multi-fold suture anchor, a multi-fold Nitinol anchor, a T-bar tissue anchor, a clip tissue anchor having two or more rigid arms, a barbed anchor, and combinations thereof; said piercing anchor connected to an attachment head comprising one or more tether stays, said tether stays selected from a loop, a cleat, an anchor, a fastener, a linkage, and combinations thereof; said attachment head connected to a tether of a heart valve repair device, said tether ranging from about 2.5 cm to 6.6 cm in length; said moderator band tissue anchor comprising a piercing anchor for piercing the moderator band or a wrapping anchor for grasping or encircling the moderator band, where the piercing anchor is sized to pierce tissue ranging from about 2.04 mm to 7.05 mm in thickness, where the wrapping anchor is sized to encircle a circumference ranging from about 6.0 mm to about 22.0 mm; and wherein the heart valve repair device comprises a replacement valve or a bypass valve selected from the group consisting of: (i) an expandable shape memory replacement valve deployed in the tricuspid annulus, or (ii) a reciprocating conduit valve, where said reciprocating conduit valve is a pliant tubular conduit mounted on a resilient annular frame and tethered within the right ventricle of the heart, wherein the pliant tubular conduit is a reciprocating mechanical member that is compressed by pressurized working fluid within the ventricle during systole.

In another preferred embodiment, the invention is directed to a medical implant, comprising an attachment device connected to the septomarginal trabecula (moderator band) of the right ventricle for securing and positioning a heart valve repair device, said attachment device sized for transcatheter delivery and deployment within the right ventricle, said attachment device having an attachment head and a moderator band tissue anchor; said attachment head connected to a tether of a heart valve repair device, said tether ranging from about 2.5 cm to 6.6 cm in length; said moderator band tissue anchor comprising a piercing anchor for piercing the moderator band or a wrapping anchor for grasping or encircling the moderator band, where the piercing anchor is sized to pierce tissue ranging from about 2.04 mm to 7.05 mm in thickness; and wherein the heart valve repair device comprises a replacement valve or a bypass valve selected from the group consisting of: (i) an expandable shape memory replacement valve deployed in the tricuspid annulus, or (ii) a reciprocating conduit valve, where said reciprocating conduit valve is a pliant tubular conduit mounted on a resilient annular frame and tethered within the right ventricle of the heart, wherein the pliant tubular conduit is a reciprocating mechanical member that is compressed by pressurized working fluid within the ventricle during systole.

In another preferred embodiment, the invention may also comprise wherein the piercing anchor is selected from the group consisting of: (i) a helical tissue anchor, (ii) a screw-type tissue anchor, (iii) a near-side T-bar tether anchor connected by a spanning tether section to a far-side T-bar tether anchor, (iv) far-side T-bar tether anchor connected by a bridge tether section to a pair of near-side tether anchors, where the near-side tether anchors are made from shape memory alloy and deform from a tubular sheath shape around the tether to a flattened, compressed disc shape by pulling the tether to draw the distal end of the sheath to the proximal end of the sheath; and (v) an arrow-shape tissue anchor that is compressed with a sheath, wherein the sheath is withdrawn to allow the memory-shape spring loaded metal arrow to open and laterally expand a pair of arrow points after the pointed sheath has penetrated the tissue to be anchored.

In another preferred embodiment, the invention may also comprise wherein the belt anchor is selected from the group consisting of: (i) a clamp-type anchor, (ii) one or more loops of material as anchor, (iii) a compression band type of anchor, (iv) a wire mesh anchor, and (v) a C-clamp type anchor.

In another preferred embodiment, the invention may also comprise wherein the tissue anchor is connected to a tether mounting ring or tether mounting hook.

In another preferred embodiment, the invention may also comprise wherein the moderator band tissue anchor comprises between 2-10 individual tissue anchors, each tissue anchor having a mounting ring or hook attached thereto.

In another preferred embodiment there is provided a method for providing an anchor mount for securing and positioning a heart valve repair device within the right ventricle, comprising the steps: (i) loading a tether platform or an attachment device within the lumen of a transcatheter delivery system and percutaneously accessing a right side of a heart; (ii) anchoring the tether platform or attachment device to the septomarginal trabecula (moderator band) of the right ventricle for securing and positioning a heart valve repair device, said tether platform or attachment device sized for transcatheter delivery and deployment within the right ventricle, said tether platform or attachment device having an attachment head and a moderator band tissue anchor; said tether platform or attachment head positioned within the right ventricle for connecting a tether of a heart valve repair device, said tether ranging from about 2.5 cm to 6.6 cm in length; said moderator band tissue anchor comprising a piercing anchor for piercing the moderator band or a wrapping anchor for grasping or encircling the moderator band, where the piercing anchor is sized to pierce tissue ranging from about 2.04 mm to 7.05 mm in thickness; and wherein the heart valve repair device comprises a replacement valve or a bypass valve selected from the group consisting of: (i) an expandable shape memory replacement valve deployed in the tricuspid annulus, or (ii) a reciprocating conduit valve, where said reciprocating conduit valve is a pliant tubular conduit mounted on a resilient annular frame and tethered within the right ventricle of the heart, wherein the pliant tubular conduit is a reciprocating mechanical member that is compressed by pressurized working fluid within the ventricle during systole.

### BRIEF DESCRIPTION OF THE SEVERAL VIEWS OF DRAWING

FIG. 1 is an illustration of a cross-section of a heart showing a prosthetic medical device tethered to a tether platform or attachment device on the septomarginal trabecula (moderator band) deployed in the right ventricle as described and claimed herein.

FIGS. 2a and 2b are illustrations of a cross-section of a heart showing percutaneous access using a transcatheter delivery via the carotid, but both carotid, femoral, sub-xyphoid, and intercostal access across the chest wall are contemplated. FIG. 2a shows a delivery catheter using an intraluminal wire to encircle the moderator band. FIG. 2b shows a circumferential anchor band secured around the longitudinal axis of the moderator band where the band is connected to an integral tether mounting ring for attaching one or more tethers of a transcatheter tricuspid replacement valve deployed for the right ventricle.

FIGS. 3(a)-(l) are a multi-feature illustration of a various sizes of unassembled top stents, cylinders, tethers, and bottom stents, and also showing a exemplary prosthetic medical device as described and claimed herein. FIG. 3(a)-(d) are illustrations of top stents, FIG. 3(e) is an illustration of a stent cover, FIG. 3(f)-(l) are illustrations of elongated flexible cylinders; illustrations of tethers are not shown as they consist of elongated filaments without need for further illustration.

FIG. 4(a) is a placement schematic for the right atrium and right ventricle and shows the channel axis, and FIG. 4(b) is an illustration of exemplary transcatheter tricuspid replacement valve tethered to the moderator band.

FIGS. 5(a) and 5(b) are illustrations showing one embodiment of the present prosthetic medical device deployed in a cross-sectional representation of a right atrium and right ventricle. FIGS. 5(a) and (b) show a time sequence of a conic-shaped intra-ventricular cylinder being compressed by systolic action of the right ventricle on the intraventricular blood. FIGS. 5a and 5b show the transcatheter tricuspid replacement valve tethered to the moderator band.

FIG. 6 is an illustration of one type of helical tissue anchor contemplated for use in anchoring as a piercing anchor in one or more locations of the moderator band.

FIG. 7 is an illustration of one type of screw-type tissue anchor contemplated for use in anchoring as a piercing anchor in one or more locations of the moderator band.

FIG. 8 is an illustration of one type of two-component tissue anchor contemplated for use in anchoring as a piercing anchor in one or more locations of the moderator band. FIG. 8 shows an anchor having a near-side T-bar tether anchor connected by a spanning tether section to a far-side T-bar tether anchor.

FIG. 9 is an illustration of another type of two-component tissue anchor contemplated for use in anchoring as a piercing anchor in one or more locations of the moderator band. FIG. 9 shows an anchor having a far-side T-bar tether anchor connected by a bridge tether section to a pair of near-side tether anchors, where the near-side tether anchors are made from shape memory alloy and deform from a tubular sheath shape around the tether to a flattened, compressed disc shape by pulling the tether to draw the distal end of the sheath to the proximal end of the sheath.

FIG. 10 is an illustration of another type of multi-component tissue anchor contemplated for use in anchoring as an encircling or wrap-type anchor for deployment in one or more locations of the moderator band. FIG. 10 shows a pair of clamping jaws having a spring located near the hinge-end where the jaw arms are connected. The jaw arms shown have a bend at each end to create a rectangular cinching space. A secondary straight arm is also shown as a mechanism for locking down the device onto the tissue once the jaws have been closed around the tissue to be captured.

FIGS. 11(a) and 11(b) are illustrations of another type of multi-component tissue anchor contemplated for use in anchoring as a piercing-type anchor for deployment in one or more locations of the moderator band. FIG. 11(b) shows an arrow-shape that is compressed with a sheath, and once the pointed sheath has penetrated the tissue to be anchored, the sheath is withdrawn to allow the memory-shape spring loaded metal arrow to open and laterally expand a pair of arrow points, as shown in FIG. 11(a). Once the tether that is connected to the anchor is pulled, the points prevent the anchor from being pulled back through the same hole that was created to deliver the point across the tissue to be anchored.

FIG. 12 is a graphic representation of the change in right ventricular pressure from diastole to systole to diastole. FIG. 12 shows the change in cross-sectional shape of the cylinder when a 2-, 3-, or 4-tether embodiment is deployed.

FIG. 13 is a graphic representation of the change in ventricular pressure from diastole to systole to diastole. FIG. 13 shows the change in cross-sectional shape of the cylinder when a 2-, 3-, or 4-tether embodiment is deployed.

FIGS. 14(a) and 14(b) are illustrations showing one embodiment of the present prosthetic medical device. FIGS. 14(a) and (b) show a time sequence of an intra-ventricular cylinder/conduit being compressed by hydro- or hemo-dynamic action of tissue that define a pressure cavity on the intracavity fluid.

FIG. 15(a)-(d) is a multi-component view of an illustration of an hourglass-shaped, three-tether, cable-type (toroid or piped-ring) top stent embodiment of the present invention. FIG. 15(a) shows an illustration of an entire device. FIG. 15(b) shows a cross-sectional view of just the frame and conduit along line C-C and shows internal surface of conduit. FIG. 15(c) shows a bottom view along line B-B and shows how the cylinder/conduit collapses to a closed position. FIG. 15(d) shows a top view along line A-A looking down the interior of the channel.

FIG. 16(a)-(c) is a multi-component view of an illustration of an hourglass-shaped, two-tether, cable-type (toroid) top stent embodiment of the present invention. FIG. 16(a) shows an illustration of an entire device. FIG. 16(b) shows a bottom view along line B-B and shows how the cylinder collapses to a closed position. FIG. 16(c) shows a top view along line A-A looking down the interior of the channel.

FIG. 17 is an illustration of another embodiment of the present device and shows a cable-style (toroidal) collar attached to an hourglass shaped cylinder/conduit that has a wide-aspect top stent/frame mounted around the cylinder. FIG. 17 shows a two tether embodiment and a low-aspect bottom-stent style anchor.

FIG. 18 is an illustration of another embodiment of the present device and shows a cable-style (toroidal) collar with a large panel attached to an hour-glass shaped conduit/cylinder that has a narrow-aspect top stent/frame mounted around the cylinder. FIG. 18 shows a two tether embodiment and a narrow-aspect bottom-stent style anchor.

FIG. 19 is an illustration of another embodiment of the present device and shows a cable-style (toroidal) collar with a large panel attached to an hour-glass shaped conduit but does not have any top stent mounted around the cylinder/conduit. FIG. 19 shows a two tether embodiment and a low-aspect bottom-stent style anchor.

FIG. 20 is an illustration of another embodiment of the present device and shows a cable-style (toroidal) collar 2 with a large panel attached to an hour-glass shaped cylinder and has a covered-frame style top stent mounted around the cylinder/conduit. FIG. 20 shows a two tether embodiment and a low-aspect bottom-stent style anchor.

FIG. 21(a)-(c) is an illustration of another embodiment of the present device and shows a vacuum-mounting feature whereby a cable-style (toroidal) collar is attached to an hourglass shaped cylinder (conduit) that has a covered-frame style top stent mounted around the cylinder, but where the top stent has a covered nitinol frame that supports a deflatable ring, wherein the deflatable ring is comprised of a toroid-shaped sealed compartment (within cover) having a valve, said sealed compartment fillable with a biocompatible liquid or gas, wherein upon removal of some or all of the biocompatible liquid or gas, the deflatable ring works in cooperation with the (non-moving) collar to compress the top spacer segment of the cylinder to a reduced height and thereby operate to seal and mount the device within a native annulus. FIGS. 21(a) and (b) shows a two tether embodiment and a low-aspect bottom-stent style anchor. FIG. 21(c) shows a cross-sectional view, sans cover.

FIGS. 22(a) and 22(b) are illustrations of another embodiment of the present device and shows in sequence an expansion-mounting feature whereby a compressed top-stent is attached to an hourglass shaped cylinder but whereby the top-stent and the bottom stent are comprised of a compressed material that is released, or of an inelastic deformable material, and thereby operate to seal and mount the device within a native annulus and native mount-area. FIGS. 22(a) and (b) show a two tether embodiment and a low-aspect bottom-stent style anchor.

FIGS. 23(a) and 23(b) are illustrations of another embodiment of the present device and shows in sequence an inflatable (or swellable)-mounting feature whereby a cable-style (toroidal) collar is attached to an hourglass shaped cylinder that has an uninflated or undeveloped top-stent attached to the hourglass shaped cylinder. FIG. 23(b) shows whereby the top-stent with polymer matrix absorbs liquid and expands, and thereby operates to seal and mount the device within a native annulus. FIGS. 23(a) and (b) show a two tether embodiment and, e.g. a tissue anchor(s).

FIGS. 24(a) and 24(b) are illustrations of another embodiment of the present device and show in sequence a thick walled cylinder being compressed by external pressure and closing the channel. FIGS. 24(a) and (b) show a two tether embodiment and a low-aspect bottom-stent style anchor.

FIG. 25(a)-(c) is an illustration of a multiple components on one embodiment of the present invention. FIG. 25(a) shows a cross-section of an open channel having two-tethers. FIG. 25(b) shows a cross-section of a compressed cylinder and closed channel having two tethers. FIG. 25(c) shows an embodiment of the prosthetic medical device having a top stent attached to a collapsible cylinder, the top stent having two contralateral annular anchors, and a two tether embodiment and a low-aspect bottom-stent style anchor.

FIG. 26 shows an embodiment of the prosthetic medical device having a top stent attached to a conic cylinder, the top stent having two contralateral annular anchors, and a three tether embodiment with two tethers attached to a low-aspect bottom-stent style anchor, and one tether attached to a tissue anchor.

FIG. 27 is an exploded view illustration of another embodiment of the present device and shows a central stent hub with aperture and having a top (apical) circumferential flange and a bottom (ventricular) circumferential flange connected to the hub, with a top toroidal inflatable ring attached to the top (apical) circumferential flange and a bottom toroidal inflatable ring attached to the bottom (ventricular) circumferential flange.

FIG. 28 is a cross-sectional side view illustration of another embodiment of the present device and shows a central stent hub with aperture and having a top (apical) circumferential flange connected to the hub, with a top toroidal inflatable ring attached to the top (apical) circumferential flange.

FIG. 29(a) is a perspective top view of another embodiment of the present device and shows a central stent hub with aperture and having a top (apical) circumferential flange connected to the hub, with a top toroidal inflatable ring attached to the top (apical) circumferential flange. FIG. 29(b) is a perspective bottom view.

FIG. 30 is an exploded view of an illustration of another embodiment of the present device and shows a central stent hub with aperture and having a top (apical) circumferential flange and a bottom tether set connected to the hub.

FIG. 31 shows catheter delivery of a compressed device to the right ventricle.

FIG. 32 is an illustration of a passive assist cage device deployed in the right atrium with pliant tubular conduit extending through the tricuspid valve annulus into the right ventricle, tethered to the moderator band.

FIG. 33 is a photographic illustration showing an view from the apex towards the tricuspid within the right ventricle and shows the moderator band in the foreground right, anterior papillary muscle left side, anterior leaflet top rear, posterior leaflet along with chordae tendinae top middle-right, and septal papillary muscle right side.

FIG. 34 is a photographic illustration showing a view from the apex-parietal side towards the tricuspid within the right ventricle and shows the moderator band in the fore-ground right, anterior papillary muscle left side, anterior leaflet top rear, posterior leaflet along with chordae tendinae top middle-right, and septal papillary muscle right side.

FIG. 35 is a photographic illustration showing a view down the tricuspid aperture towards the right ventricular apex and shows the moderator band in the center, anterior papillary muscle right side, and septal papillary muscle left side.

FIG. 36 is a photographic illustration showing a downward view across the right ventricle from the septal wall towards the parietal/apex and shows the moderator band in the center, anterior papillary muscle behind, and trabeculae carneae convolutions left side.

### DETAILED DESCRIPTION OF THE INVENTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Rather, these embodiments are provided so that this disclosure will be thorough and complete, and will fully convey the scope of the invention to those skilled in the art. Like numbers refer to like elements throughout. As used herein the term “and/or” includes any and all combinations of one or more of the associated listed items.

The terminology used herein is for the purpose of describing particular embodiments only and is not intended to limit the full scope of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal subparts. As will be understood by one skilled in the art, a range includes each individual member.

### Definitions

Belt or Band—refers to a flat, thin strip or loop of material that put around something, e.g. the septomarginal trabecula, i.e. a flat, thin strip or loop of material that encircles the (anatomical) ‘moderator band’ within the right ventricle, i.e. a surgically places loop encircling an elongated band of native tissue that traverses or crosses the native right ventricle. Contemplated as within the scope of the invention, the band may also be configured as a band, belt, wrap, girdle, strap, tape, ring, sheath, cage, collar, scaffold, or a combination of two or more structures. In the present invention, this belt/band/collar is used as a platform for attaching a tether to an implanted medical device, and in particular prosthetic valves, rings, clips, and so forth, for treating diseases and problems with the tricuspid and/or pulmonary valve. The band is constructed of a mesh, fabric, braid, coil, windings of suture, or combinations thereof. The belt or band materials may be biocompatible tissue, metal e.g. Nitinol wire or mesh, polymers, and combinations thereof.

Platform refers to a belt or band or collar device attached to the septomarginal trabecula, with additional anchoring components for securing a tether. Contemplated as within the scope of the invention, the platform may configured as a base, mount, footing, frame, pedestal, seat, and support.

Tether anchor refers to a component attached to the moderator band collar for securing a tether. Tether anchor includes a loop, linkage, cleat, fastener, and stay. A tether anchor is similar to a tissue anchor in that both are attachments for tethers, however, a tissue anchor includes features for piercing and permanently connecting to tissue and in particular to intraventricular myocardium. Also contemplated as within the scope of the invention is the use of multiple tether anchors.

Body channel—defined as a conduit or vessel within the body. Of course, the particular application of the prosthetic heart valve determines the body channel at issue. An aortic valve replacement, for example, would be implanted in, or adjacent to, the aortic annulus. Likewise, a tricuspid or mitral valve replacement will be implanted at the tricuspid or mitral annulus. Certain features of the present invention are particularly advantageous for one implantation site or the other. However, unless the combination is structurally impossible, or excluded by claim language, any of the heart valve embodiments described herein could be implanted in any body channel.

Bore—The inside diameter of the cylinder tube.

Bypass—A secondary passage for fluid flow.

Discharge hose, or discharge tubing—also called a backwash hose, lay-flat hose. A flexible cylinder or tubing that expands to cylindrical shape (rounded cross-section) due to internal hydraulic pressure when filled with fluid, and that collapses or flattens or seals when the internal hydraulic pressure is reduced by removing or lessening the amount of fluid.

Displacement—The volume of fluid displaced by one complete stroke or revolution.

Ejection fraction is a measurement of the percentage of blood leaving your heart each time it contracts. During each heartbeat pumping cycle, the heart contracts and relaxes. When your heart contracts, it ejects blood from the two pumping chambers (ventricles).

As a point of further definition, the term “expandable” is used herein to refer to a component of the heart valve capable of expanding from a first, delivery diameter to a second, implantation diameter. An expandable structure, therefore, does not mean one that might undergo slight expansion from a rise in temperature, or other such incidental cause. Conversely, “non-expandable” should not be interpreted to mean completely rigid or a dimensionally stable, as some slight expansion of conventional “non-expandable” heart valves, for example, may be observed.

Force—A push or pull acting upon a body. In a hydraulic cylinder, it is the product of the pressure on the fluid, multiplied by the effective area of the cylinder piston.

Prosthetic Valve

The term prosthesis or prosthetic encompasses both complete replacement of an anatomical part, e.g. a new mechanical valve replaces a native valve, as well as medical devices that take the place of and/or assist, repair, or improve existing anatomical parts, e.g. native valve is left in place. For mounting within a passive assist cage, the invention contemplates a wide variety of (bio)prosthetic artificial heart valves. Contemplated as within the scope of the invention are ball valves (e.g. Starr-Edwards), bileaflet valves (St. Jude), tilting disc valves (e.g. Bjork-Shiley), stented pericardium heart-valve prosthesis' (bovine, porcine, ovine) (Edwards line of bioprostheses, St. Jude prosthetic valves), as well as homograft and autograft valves. For bioprosthetic pericardial valves, it is contemplated to use bioprosthetic aortic valves, bioprosthetic mitral valves, bioprosthetic tricuspid valves, and bioprosthetic pulmonary valves.

Septomarginal Trabecula Aka Moderator Band

The septomarginal trabecula of the right ventricle, originally termed the moderator band because it was thought to limit the lateral expansion of the chamber, is a muscular thickening extending from the interventricular septum to the base of the anterior papillary muscle. One of the main functions of the septomarginal trabecula is to convey the right branch of the atrioventricular bundle of the conducting system. The septomarginal trabecula also functions to form the anteroinferior border between the superior, smooth outflow tract of the ventricle and the trabeculated inflow tract. At its septal attachment, it may be continuous with the supraventricular crest.

Frame—Stent Structure

Preferably, the frame is made from superelastic metal wire, such as Nitinol (TM) wire or other similarly functioning material. The material may be used for the frame/stent, for the collar, and/or for the apex anchor/bottom stent. It is contemplated as within the scope of the invention to use other shape memory alloys such as Cu—Zn—Al—Ni alloys, Cu— Al—Ni alloys, as well as polymer composites including composites containing carbon nanotubes, carbon fibers, metal fibers, glass fibers, and polymer fibers. It is contemplated that the frame/top stent, collar, and bottom stent may be constructed as a braided stent or as a laser cut stent. Such stents are available from any number of commercial manufacturers, such as Pulse Systems. Laser cut stents are preferably made from Nickel-Titanium (Nitinol (TM)), but also without limitation made from stainless steel, cobalt chromium, titanium, and other functionally equivalent metals and alloys, or Pulse Systems braided stent that is shape-set by heat treating on a fixture or mandrel.

One key aspect of the stent design is that it be compressible and when released have the stated property that it return to its original (uncompressed) shape. This requirement limits the potential material selections to metals and plastics that have shape memory properties. With regards to metals, Nitinol has been found to be especially useful since it can be processed to be austhenitic, martensitic or super elastic. Martensitic and super elastic alloys can be processed to demonstrate the required compression features.

Laser Cut Stent

One possible construction of the stent envisions the laser cutting of a thin, isodiametric Nitinol tube. The laser cuts form regular cutouts in the thin Nitinol tube.

Secondarily the tube is placed on a mold of the desired shape, heated to the Martensitic temperature and quenched. The treatment of the stent in this manner will form a stent or stent/cuff or atrial sealing gasket that has shape memory properties and will readily revert to the memory shape at the calibrated temperature.

Braided Wire Stent

A stent can be constructed utilizing simple braiding techniques. Using a Nitinol wire—for example a 0.012″ wire—and a simple braiding fixture, the wire is wound on the braiding fixture in a simple over/under braiding pattern until an isodiametric tube is formed from a single wire. The two loose ends of the wire are coupled using a stainless steel or Nitinol coupling tube into which the loose ends are placed and crimped. Angular braids of approximately 60 degrees have been found to be particularly useful. Secondarily, the braided stent is placed on a shaping fixture and placed in a muffle furnace at a specified temperature to set the stent to the desired shape and to develop the martensitic or super elastic properties desired.

Tethers—The tethers are made from surgical-grade materials such as biocompatible polymer suture material. Non-limiting examples of such material include ultra high-molecular weight polyethylene (UHMWPE), 2-0 exPFTE(polytetrafluoroethylene) or 2-0 polypropylene. In one embodiment the tethers are inelastic. It is also contemplated that one or more of the tethers may optionally be elastic to provide an even further degree of compliance of the valve during the cardiac cycle.

Tines-Anchors—Tines/Barbs

The device can be seated within the valvular annulus through the use of tines or barbs. These may be used in conjunction with, or in place of one or more tethers. The tines or barbs are located to provide attachment to adjacent tissue. Tines are forced into the annular tissue by mechanical means such as using a balloon catheter. In one non-limiting embodiment, the tines may optionally be semi-circular hooks that upon expansion of the stent body, pierce, rotate into, and hold annular tissue securely.

Tissue—The tissue used herein is a biological tissue that is a chemically stabilized pericardial tissue of an animal, such as a cow (bovine pericardium) or sheep (ovine pericardium) or pig (porcine pericardium) or horse (equine pericardium). Preferably, the tissue is bovine pericardial tissue. Examples of suitable tissue include that used in the products Duraguard®, Peri-Guard®, and Vascu-Guard®, all products currently used in surgical procedures, and which are marketed as being harvested generally from cattle less than 30 months old. Other patents and publications disclose the surgical use of harvested, biocompatible animal thin tissues suitable herein as biocompatible “jackets” or sleeves for implantable stents, including for example, U.S. Pat. No. 5,554,185 to Block, U.S. Pat. No. 7,108,717 to Design & Performance-Cyprus Limited disclosing a covered stent assembly, U.S. Pat. No. 6,440,164 to Scimed Life Systems, Inc. disclosing a bioprosthetic valve for implantation, and U.S. Pat. No. 5,336,616 to LifeCell Corporation discloses acellular collagen-based tissue matrix for transplantation.

In one preferred embodiment, the conduit may optionally be made from a synthetic material such a polyurethane or polytetrafluoroethylene.

Where a thin, durable synthetic material is contemplated, e.g. for a covering, synthetic polymer materials such expanded polytetrafluoroethylene or polyester may optionally be used. Other suitable materials may optionally include thermoplastic polycarbonate urethane, polyether urethane, segmented polyether urethane, silicone polyether urethane, silicone-polycarbonate urethane, and ultra-high molecular weight polyethylene. Additional biocompatible polymers may optionally include polyolefins, elastomers, polyethylene—glycols, polyethersulphones, polysulphones, polyvinylpyrrolidones, polyvinylchlorides, other fluoropolymers, silicone polyesters, siloxane polymers and/or oligomers, and/or polylactones, and block co-polymers using the same.

Examples of preferred embodiments of the reciprocating pressure conduit (RPC) valve include the following details and features.

### Example 1

One preferred embodiment of a moderator band device is a strap or collar made of polymer fabric mounted around the moderator band and having at least two tether attachment members.

### Example 2

Another preferred embodiment of a moderator band device is a strap or collar made of a plurality of suture windings mounted around the moderator band and having at least two tether attachment members.

### Example 3

Another preferred embodiment of a moderator band device is a strap or collar made of a Nitinol coil mounted around the moderator band and having at least two tether attachment members.

### Example 4

Another preferred embodiment of a moderator band device is a strap or collar made of a Nitinol mesh mounted around the moderator band and having at least two tether attachment members.

### Example 5

One preferred embodiment of a moderator band-tethered transcatheter valve is a heart valve substitute comprising a pliant tubular conduit that is mounted on a resilient annular or sub-annular frame and that is tethered to a non-perforating anchor within the right ventricle of the heart, wherein the pliant tubular conduit is a reciprocating mechanical member that is compressed by pressurized working fluid, blood, within the ventricle during systole. Importantly, this heart valve substitute has no leaflets and does not have a traditional valve configuration. Additionally, the device can be delivered to the ventricle compressed within a catheter, and expelled from the catheter to be deployed without open heart surgery.

### Example 6

In another preferred embodiment of a moderator band-tethered transcatheter valve, comprises: (i) an elongated flexible cylinder defining a channel therein, said channel having a volume that ranges from 1.57 mL-18.84 mL, said cylinder having an average radius of 4.0-16.5 mm and an average height of 20-60 mm, said cylinder comprised of decellularized pericardium, said cylinder having top end, a bottom end, an internal surface, and an external surface, said cylinder is compressible under a pressure of 100-160 mm Hg on the external surface to close the channel, and said cylinder is expandable under a pressure of 40-80 mm Hg on the internal surface to open the channel; (ii) a one-piece, laser-cut, expandable nitinol top stent, said top stent attached to the top end of the cylinder, said top stent shaped as a conic frustum when expanded and defining a top stent channel therein, said conic frustum having a side wall, a top aperture, and a bottom aperture, said side wall having an average side length of 5-20 mm, said top aperture having an average expanded diameter of 30-35 mm, said bottom aperture having having an average expanded diameter of 40-60 mm, said top stent having a cover, said cover connected with the cylinder wherein the channel of the cylinder is in communication with the top stent channel; and (iii) a one-piece, laser-cut, expandable nitinol bottom stent, said bottom stent having a top end, a bottom end, and a side wall, said top end of the bottom stent having from 2-5 tethers attached to the bottom end of the cylinder, said bottom stent having an average expanded diameter of 20-35 mm.

### Example 7

In additional preferred embodiments of a moderator band-tethered transcatheter valve, there are additional features, including: (1) wherein the cylinder is shaped as a conic cylinder, said top end having a diameter of 30-35 mm and said bottom end having a diameter of 8-20 mm; (2) where the top stent cover is comprised of polyethylene terephthalate, decellularized pericardium, or a layered combination thereof; (3) wherein the top end of the cylinder comprises, in order, a top edge connected to a top spacer segment that is connected to a top stent mounting segment, wherein the top edge has an collar mounted around the circumference of the top edge, said collar arranged as a flexible, semi-rigid, substantially flat panel or flat disk and having an average diameter of 30-60 mm, said collar having a nitinol frame covered with polyethylene terephthalate, decellularized pericardium, or a layered combination thereof, wherein the top spacer segment of the cylinder has a height from 5-20 mm, and wherein the top stent is mounted circumferentially around the top stent mounting segment of the cylinder; (4) wherein the collar has one or more tissue anchors arranged along the circumference of the collar; (5) wherein the nitinol frame of the collar supports a gel ring, wherein the gel ring is comprised of an expandable material enclosed within an outer sealing membrane, wherein the expandable material is a swellable powder within a polymeric matrix, a swellable polymeric matrix, or a swellable polymeric liquid; (6) wherein the deflatable ring is comprised of a toroid-shaped sealed compartment having a valve, said sealed compartment fillable with a biocompatible liquid or gas, wherein upon removal of some or all of the biocompatible liquid or gas, the deflatable ring has a reduced diameter, and wherein upon removal of some or all of the biocompatible liquid or gas, the top spacer segment of the cylinder has a reduced height and the collar is compressed in the direction of the top stent; (7) wherein the top stent has one or more tissue anchors arranged along the side wall of the top stent; (8) wherein the bottom stent has one or more tissue anchors arranged along the side wall of the bottom stent; (9) wherein the cylinder has an hourglass (hyperboloid) shape from top end to bottom end; (10) wherein the bottom end of the cylinder is sealed, and wherein the cylinder has one or more perforations in a mid-segment side wall of the cylinder; (11) wherein the top stent comprises a central stent hub with aperture and having a top circumferential flange and a bottom circumferential flange connected to the hub, with a top toroidal inflatable ring attached to the top circumferential flange and a bottom toroidal inflatable ring attached to the bottom circumferential flange; and (12) wherein the top stent comprises a threaded structure on an exterior surface of the stent, wherein the threaded structure allows for a simple circular screw-type deployment of the device into a native annulus to aid in sealing and sizing of the top stent into the native annulus.

### Example 8—Method

In another preferred embodiment of the invention, there is also provided a method of controlling flow of bodily fluid within an enclosed cavity of a human body, said enclosed cavity having a reciprocating pressure differential, the method comprising the steps: (i) delivering and attaching a moderator band platform, consisting of a band or collar having attachment members, around the septomarginal trabecula; (ii) delivering a prosthetic medical device to the enclosed cavity within the human body; and (iii) tethering the prosthetic medical device to the moderator band platform.

### Example 9

In another preferred method the prosthetic medical device is a reciprocating pressure conduit (RPC) valve as described herein, and includes additional method steps of:

(iv) arranging the prosthetic medical device of claim 1 whereby the cylinder and cylinder channel are arranged parallel to a flow of fluid entering the enclosed cavity; (v) expanding the top stent within an entrance to the enclosed cavity to mount the top end of the cylinder within the entrance, and whereby the side wall of the top stent applies an axial compression force and seals the entrance; (vi) anchoring the cylinder to the moderator valve within the enclosed cavity to anchor the bottom end of the cylinder; wherein bodily fluid arriving at the enclosed cavity is diverted into the channel of the cylinder; wherein the reciprocating pressure differential comprises a low pressure state and a high pressure state; wherein bodily fluid flows into the channel to the enclosed cavity during the low pressure state, and wherein bodily fluid is prevented from flowing into the channel to the enclosed cavity during the high pressure state, wherein the high pressure state exerts a force on the external surface of the cylinder and collapses the reversibly collapses the channel.

### Example 10

In another preferred embodiment, the heart valve substitute comprises a pliant tubular conduit that is mounted on a resilient annular or sub-annular frame, the conduit is tethered to a moderator band anchor platform for deployment within a right ventricle of a heart, wherein the pliant tubular conduit is a reciprocating mechanical member that is compressed by pressurized working fluid within the ventricle during a high pressure phase of the heart (systole).

### Example 11

In another preferred embodiment of a moderator band-tethered transcatheter valve, the medical prosthesis is a heart valve substitute comprising a pliant tubular conduit that is mounted on a resilient expandable passive assist cage, the cage is deployed within an atrial or ventricular chamber of a heart, wherein the pliant tubular conduit is a reciprocating mechanical member that is compressed by pressurized working fluid within the ventricle during a high pressure phase of the heart (systole) and opened by lower pressure working fluid within the ventricle during a low pressure phase of the heart (diastole). In this example, the cage defines an interior cavity and the conduit is mounted within the cavity, or the conduit is mounted outside of the cavity.

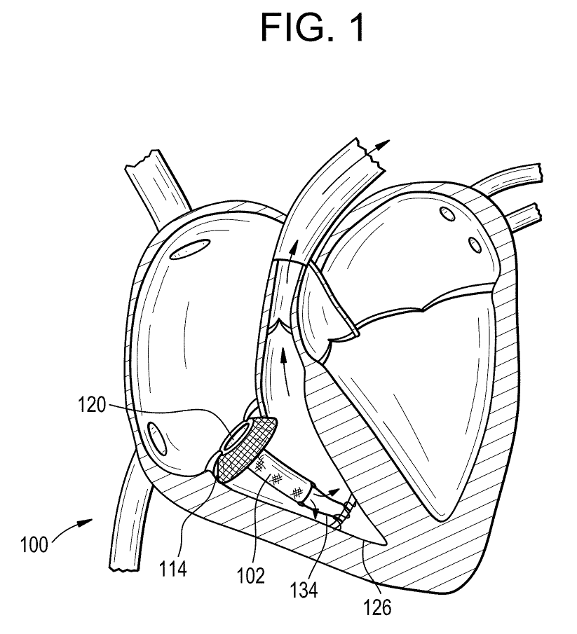

Referring now to the drawings, FIG. 1 is an illustration of a cross-section of a heart showing a prosthetic medical device as described and claimed herein deployed in the right ventricle. FIG. 1 shows a moderator-band tethered transcatheter valve/prosthetic medical device 100 comprised of top stent/resilient subannular frame 114 supporting the elongated flexible cylinder/pliant tubular conduit 102. Tethers 134 connect conduit 102 to septomarginal trabecula anchor platform (moderator band anchor platform) 126. Frame (or stent) 114 is anchored below the native tricuspid valve by one or more suitable anchor devices such as surgical clips, clamps, and so forth. Frame 114 is a self-expanding or balloon expandable structure that holds the device within the native annulus and also prevents the device from being ejected into the right atrium during systole. Frame 114, anchor 126 and tethers 134 may be constructed, in whole or in part, of suitable metal, polymeric, or composite materials including nickel-titanium alloy, cobalt-chromium alloy, high cycle fatigue tolerant polymers including composites containing glass fiber, polymer fiber, carbon fiber, metal fiber, carbon nanotube fiber, and composites containing polymer filler materials.

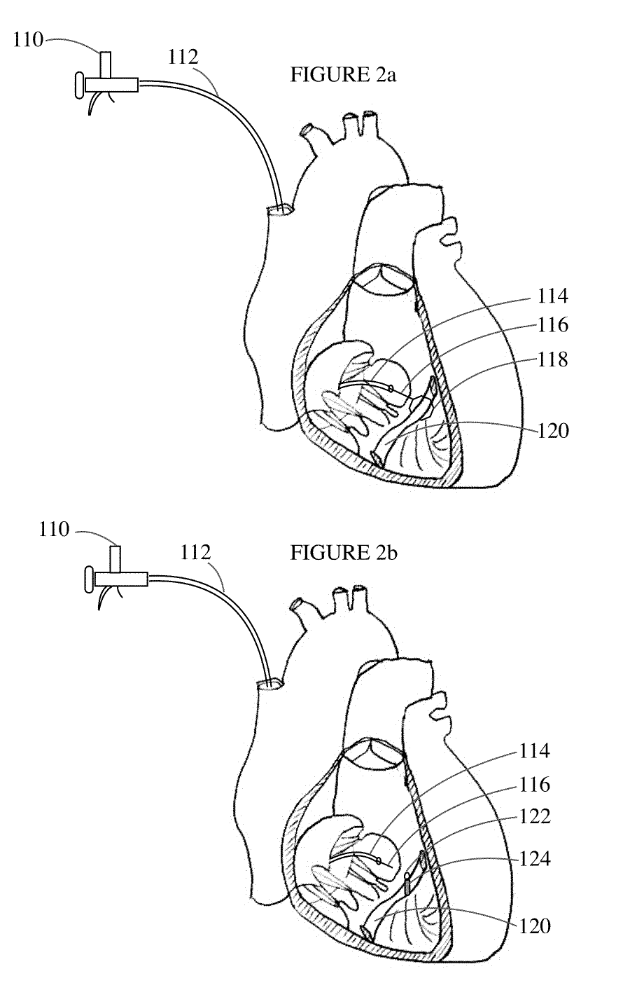

FIGS. 2(a) and (b) are illustrations of a cross-section of a heart showing percutaneous access using a transcatheter delivery via the carotid, but both carotid, femoral, sub-xyphoid, and intercostal access across the chest wall are contemplated. Transcatheter delivery device has actuating handle 109 and delivery catheter 111 (external). FIG. 2a shows a delivery catheter 115 within the right atrium (RA) using a catheter tool 117 or an intraluminal wire to encircle the moderator band 121 with a cincture or encircling suture member 119. FIG. 2b shows a circumferential anchor band 123 secured around the longitudinal axis of the moderator band 121 where the band 123 is connected to an integral tether mounting ring 122 for attaching one or more tethers (134 of 2a) of a transcatheter tricuspid replacement valve deployed for the right ventricle.

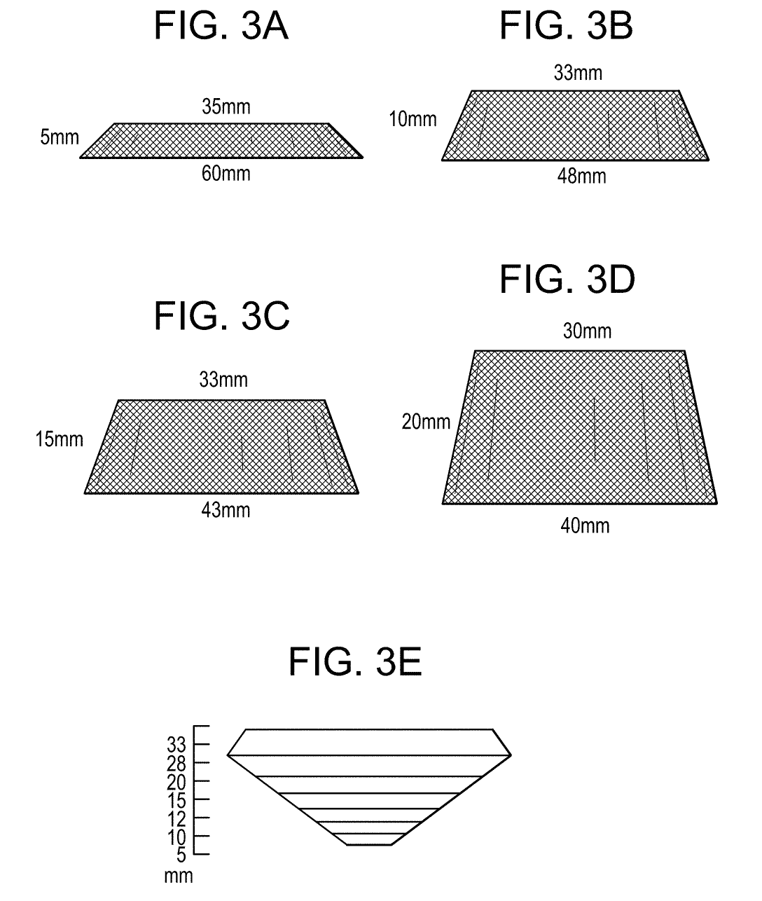

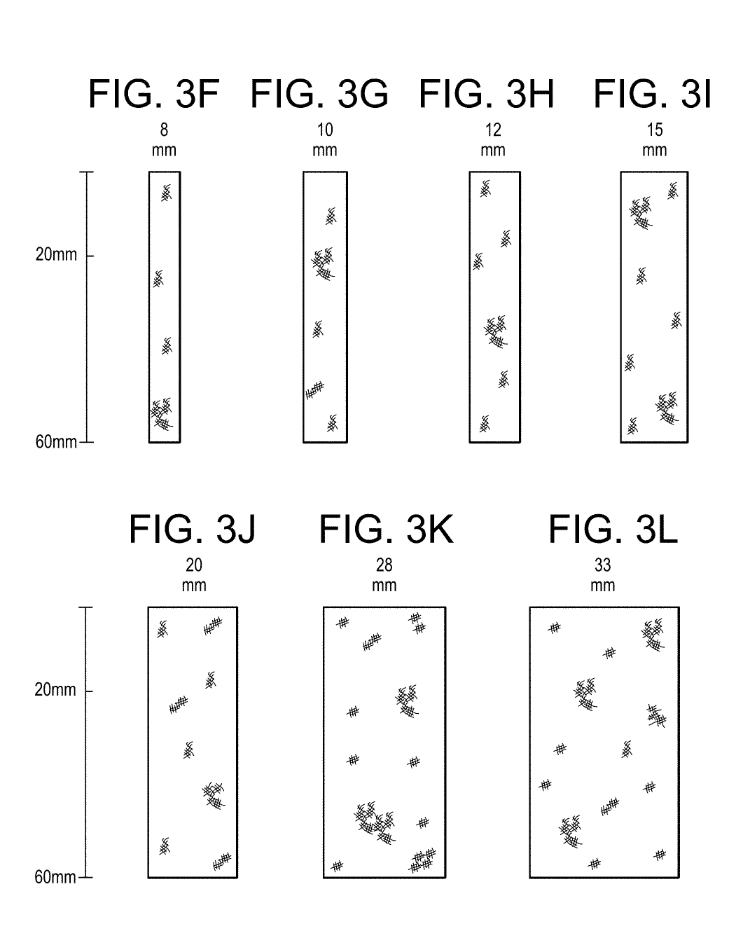

FIG. 3 is a multi-feature illustration of a various sizes of unassembled top stents 114, and cylinders 102. FIG. 3(a)-(d) are illustrations of top stents 114, FIG. 3(e) is an illustration of a stent cover, FIG. 3(f)-(l) are illustrations of elongated flexible cylinders 102; illustrations of tethers are not shown as they consist of elongated filaments without need for further illustration.

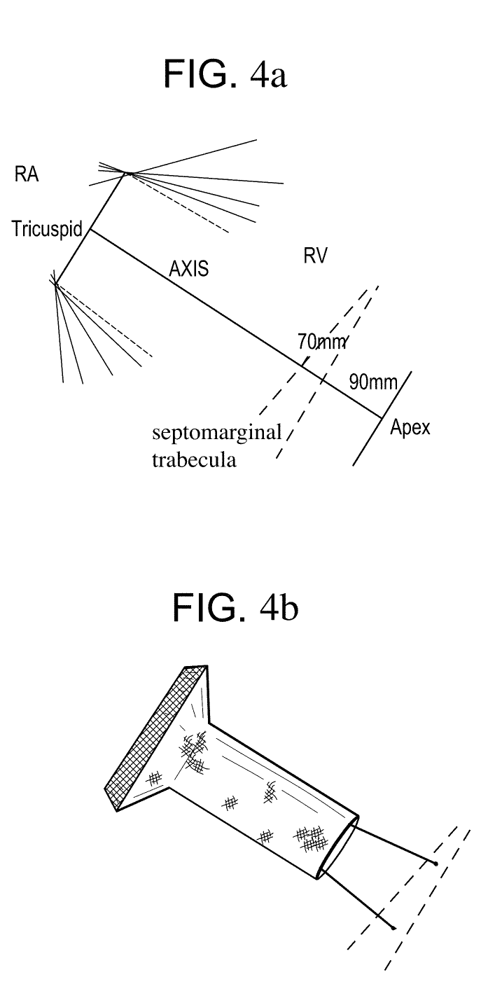

FIG. 4(a) is a placement schematic for the right atrium and right ventricle and shows the channel axis, and FIG. 4(b) is an illustration of exemplary transcatheter tricuspid replacement valve 114, 102 tethered 134 to the moderator band 121.

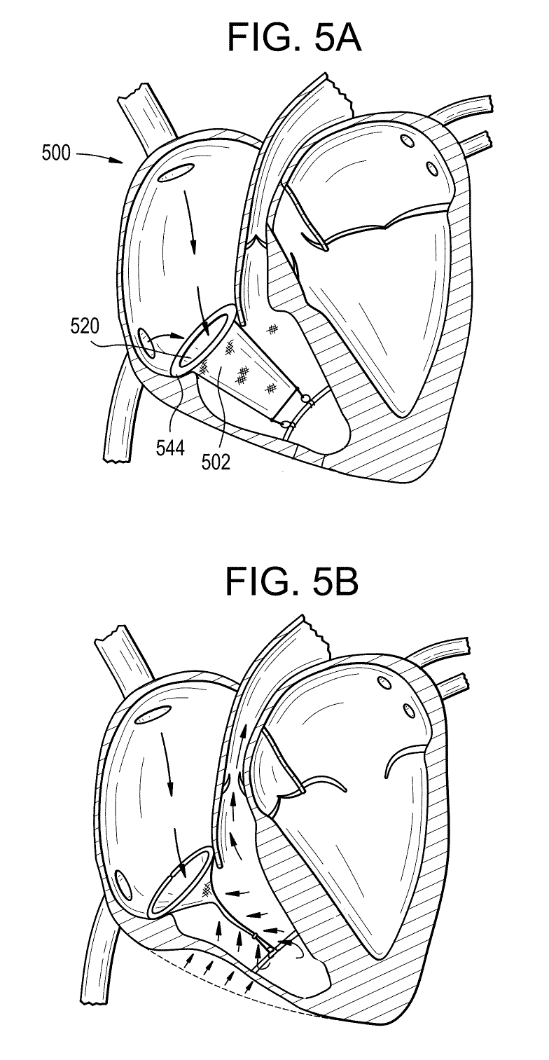

FIGS. 5(a) and 5(b) are illustrations showing one embodiment of the present prosthetic medical device 500 deployed in a cross-sectional representation of a right atrium and right ventricle. FIGS. 5(a) and (b) show a time sequence of a conic-shaped intra-ventricular cylinder/conduit 502 being compressed by systolic action of the right ventricle on the intraventricular blood. FIG. 5 shows conic cylinder shaped conduit 502 mounted to supra-annular collar 544 with collar aperture 520 leading down the conduit lumen to tether 534 connected to apical moderator band platform anchor 526 attached to the septomarginal trabecula 521. FIG. 5A also shows linkage member 528 connecting the collar/band 526 to the tether(s) 534.

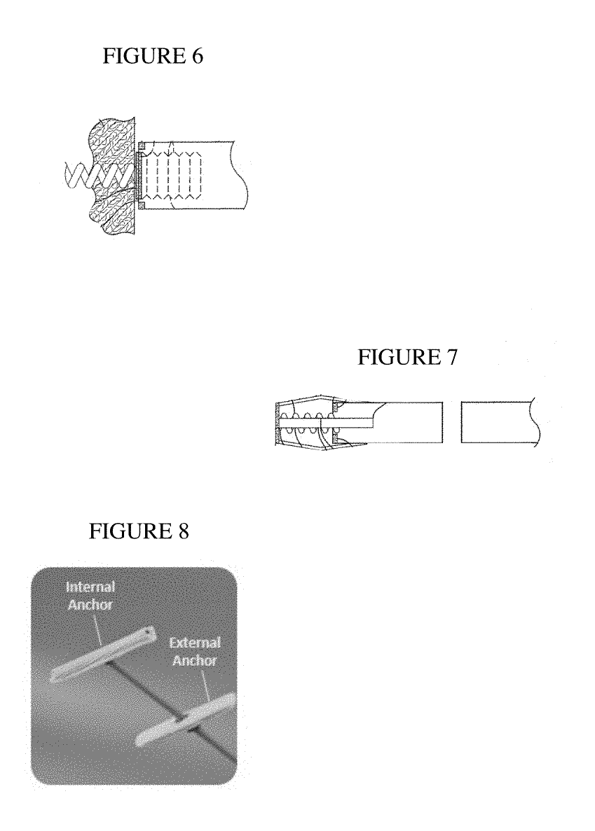

FIG. 6 is an illustration of one type of helical tissue anchor contemplated for use in anchoring as a piercing anchor 130 in one or more locations of the moderator band. FIG. 6 shows piercing anchor 130 having helical member 131 advanced into tissue after being released or fed by release/feed mechanism 132 from cavity 133. This piercing anchor is delivered within catheter sheath 135. Release of the helical member 131 from the cavity 133 may be as a spring mechanism. Alternatively, the helical member 131 may be fed from the cavity 133 using a screw mechanism. Spring and screw mechanisms are controlled by anchor manipulation tool 137 which extends external the patient to the catheter control handle. Tethers may be pre-attached to the piercing anchor or the piercing anchor may be have an integral loop, hook, cleat, or clip for attaching a tether.

FIG. 7 is an illustration of one type of screw-type tissue anchor 140 contemplated for use in anchoring as a piercing anchor in one or more locations of the moderator band. Screw member 141 is driven by (spring) release or feeder mechanism 142 actuated by anchor manipulation tool 147, housed within anchor sheath 145.

FIG. 8 is an illustration of one type of two-component tissue anchor 160 contemplated for use in anchoring as a piercing anchor in one or more locations of the moderator band. FIG. 8 shows an anchor 160 having a near-side T-bar tether anchor 165 connected by a spanning tether section 166 to a far-side T-bar tether anchor 164. T-bar anchors may be constructed using an elongated tether 161 that is threaded through tissue using the anchor tool 162 which extends from catheter 163. Using a compression or series of accordion-type compressions the elongated tether 161 is formed into T-bar tethers 164, 165 for anchoring to the tissue. Also contemplated within the scope of the invention is the deployment of a single T-bar tether anchor.

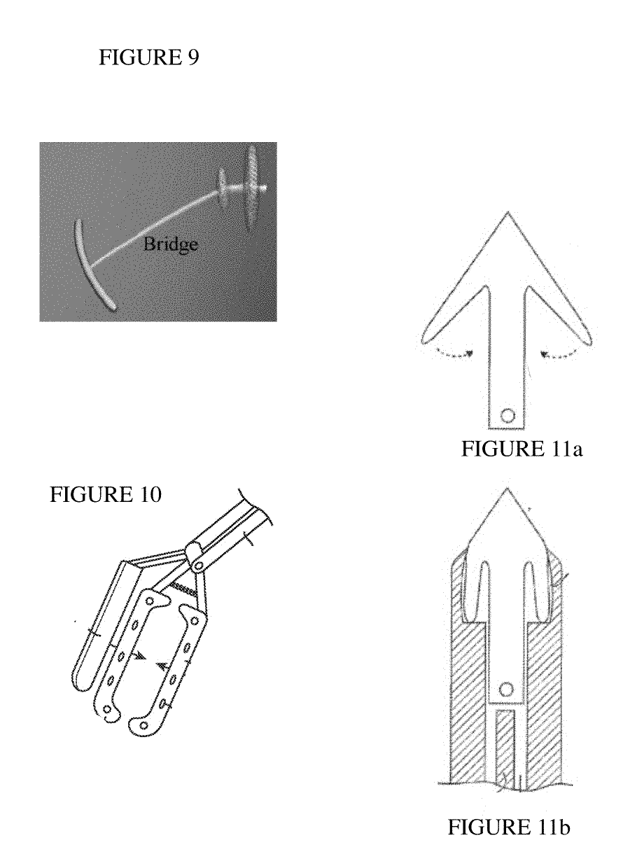

FIG. 9 is an illustration of another type of two-component tissue anchor 170 contemplated for use in anchoring as a piercing anchor in one or more locations of the moderator band. FIG. 9 shows an anchor 170 having a far-side T-bar tether 174 anchor connected by a bridge tether section 176 to a pair of near-side tether anchors 175, 171, where the near-side tether anchors are made from shape memory alloy and deform from a tubular sheath shape around the tether to a flattened, compressed disc shape by pulling the tether to draw the distal end of the sheath to the proximal end of the sheath.

FIG. 10 is an illustration of another type of multi-component tissue anchor 180 contemplated for use in anchoring as an encircling or wrap-type anchor for deployment in one or more locations of the moderator band. FIG. 10 shows a pair of clamping jaws or brackets 184, 185 having a spring 189 located near the hinge-end where the first and second control arms 182, 183 are connected. The bracket members 184, 185 shown have a bend at each end to create a rectangular cinching space. A third control arm 186 is also shown supporting bracket locking sleeve 187 as a mechanism for locking down the device (brackets) onto the tissue once the bracket jaws have been closed around the tissue to be captured.

FIG. 11 is an illustration of another type of multi-component barb tissue anchor 190 contemplated for use in anchoring as a piercing-type anchor for deployment in one or more locations of the moderator band. FIG. 11 shows an arrow-shape anchor consisting of a shaft 193 mounted on a anchor tool 191 and one or more barbs 192 that are compressed with a sheath 195 of a delivery catheter 194. Once the penetrating point 196 and sheath 195 has penetrated the tissue to be anchored, the sheath 195 is withdrawn to allow the memory-shape spring loaded metal arrow to open and laterally expand a pair of barbs or arrow points 192. Once the tether that is connected to the anchor is pulled, the points prevent the anchor from being pulled back through the same hole that was created to deliver the penetrating point 196 across the tissue to be anchored.

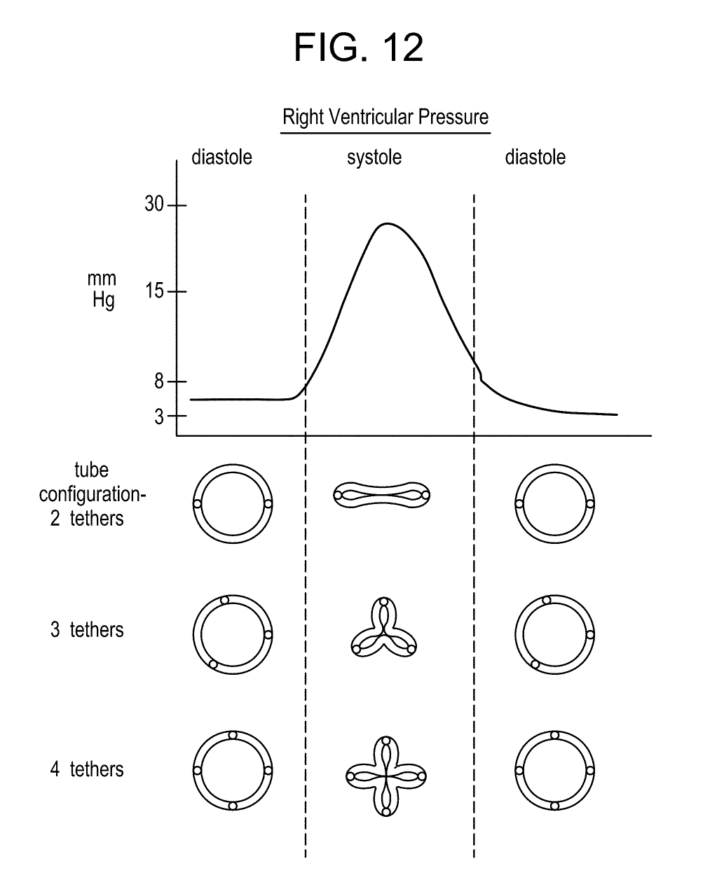

FIG. 12 is a graphic representation of the change in right ventricular pressure from diastole to systole to diastole. FIG. 12 shows the change in cross-sectional shape of the cylinder when a 2-, 3-, or 4-tether embodiment is deployed. FIG. 12 shows pressure in mm Hg along the Y-axis and the phase of the heart cycle along the X-axis. For the right ventricle, diastole can be, for example, about 5 mm Hg. However, during right ventricular systole, the intraventicular pressure can rise to around 30 mm Hg., closing the conduit. FIG. 12 shows how in a two-tether embodiment, the conduit collapses to form a horizontal bi-fold seal. FIG. 12 shows how in a three-tether embodiment, the conduit collapses to form a triangular tri-fold seal. FIG. 12 also shows how in a four-tether embodiment, the conduit collapses to form a cross-shaped four-fold seal.

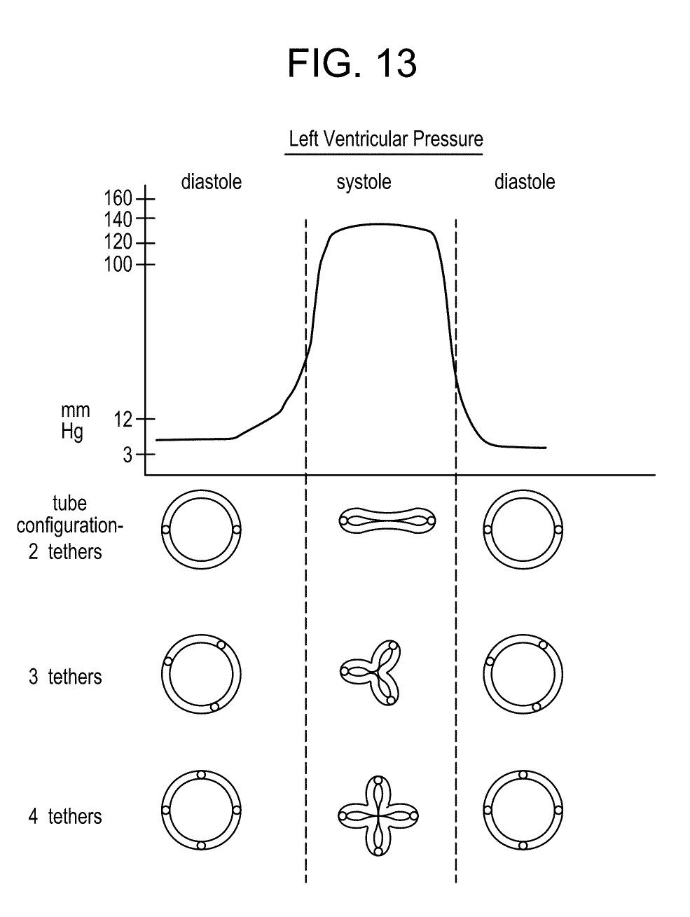

FIG. 13 is a graphic representation of the change in ventricular pressure from diastole to systole to diastole. FIG. 13 shows the change in cross-sectional shape of the cylinder when a 2-, 3-, or 4-tether embodiment is deployed. FIG. 13 shows pressure in mm Hg along the Y-axis and the phase of the heart cycle along the X-axis. For the ventricle, diastole can be, for example, as low as 8 mm Hg. However, during ventricular systole, the intraventicular pressure can rise up to 160 mm Hg. or higher, closing the conduit. FIG. 13 shows how in a two-tether embodiment, the conduit collapses to form a horizontal bi-fold seal. FIG. 13 shows how in a three-tether embodiment, the conduit collapses to form a triangular tri-fold seal. FIG. 13 also shows how in a four-tether embodiment, the conduit collapses to form a cross-shaped four-fold seal.

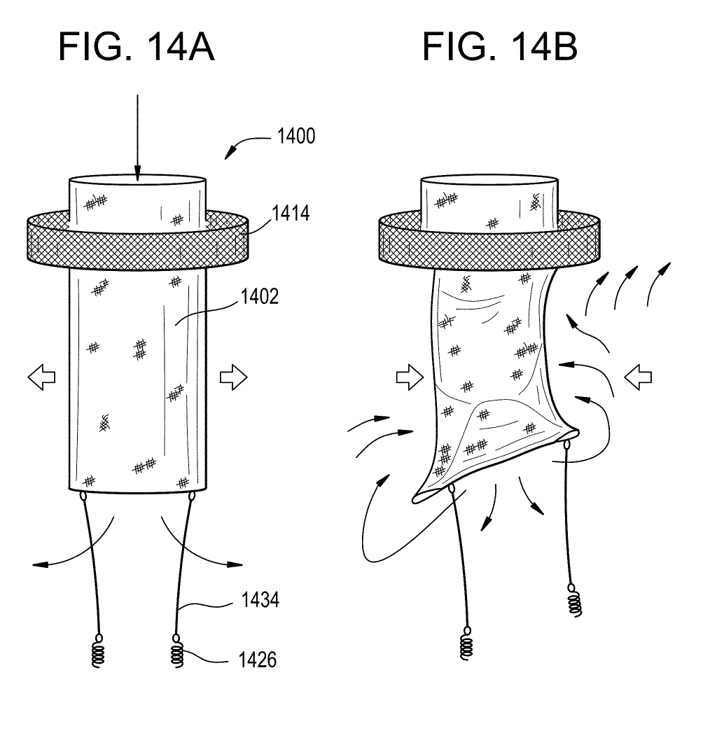

FIGS. 14(a) and 14(b) are illustrations showing one embodiment of the present prosthetic medical device 1400. FIGS. 14(a) and (b) show a time sequence of an intra-ventricular cylinder/conduit being compressed by hydro- or hemo-dynamic action of tissue that define a pressure cavity on the intracavity fluid. FIGS. 14(a) and (b) also illustrate a simple device having only a frame/stent 1414 and cylinder/conduit 1402 having two tethers 1434 attached to tissue anchors 1426.

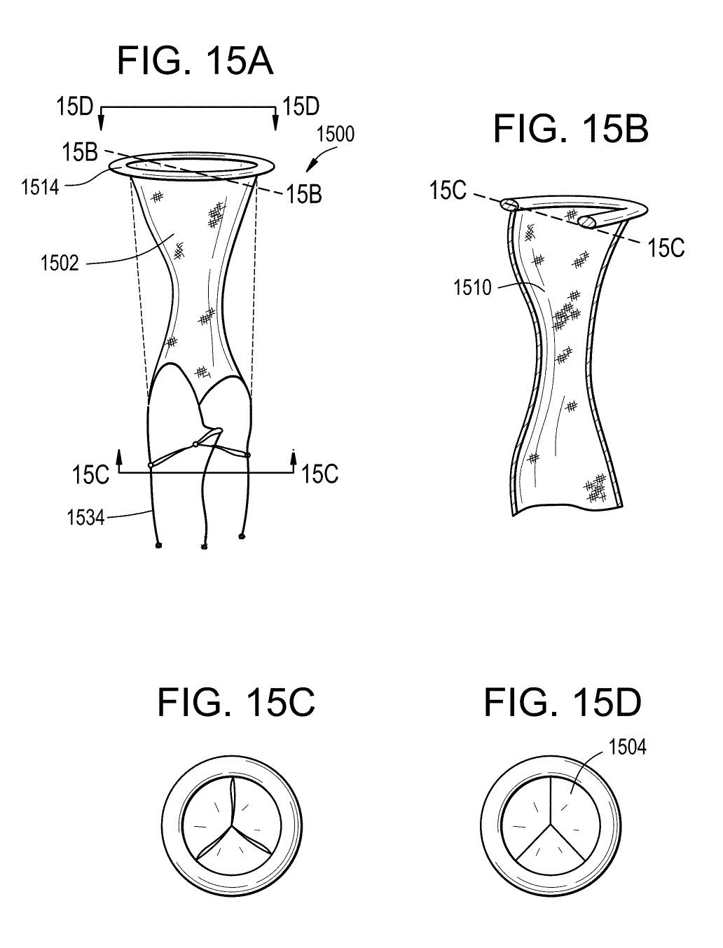

FIG. 15(a)-(d) is a multi-component view of an illustration of an hourglass-shaped, three-tether 1534, cable-type (toroid or piped-ring) top stent 1514 embodiment of the present invention 1500 FIG. 15(a) shows an illustration of an entire device. FIG. 15(b) shows a cross-sectional view of just the frame 1514 and conduit 1502 along line C-C and shows internal surface 1510 of conduit. FIG. 15(c) shows a bottom view along line B-B and shows how the cylinder/conduit 1502 collapses to a closed position. FIG. 15(d) shows a top view along line A-A looking down the interior of the channel 1504. FIG. 15 also shows bottom stent/anchor 1526.

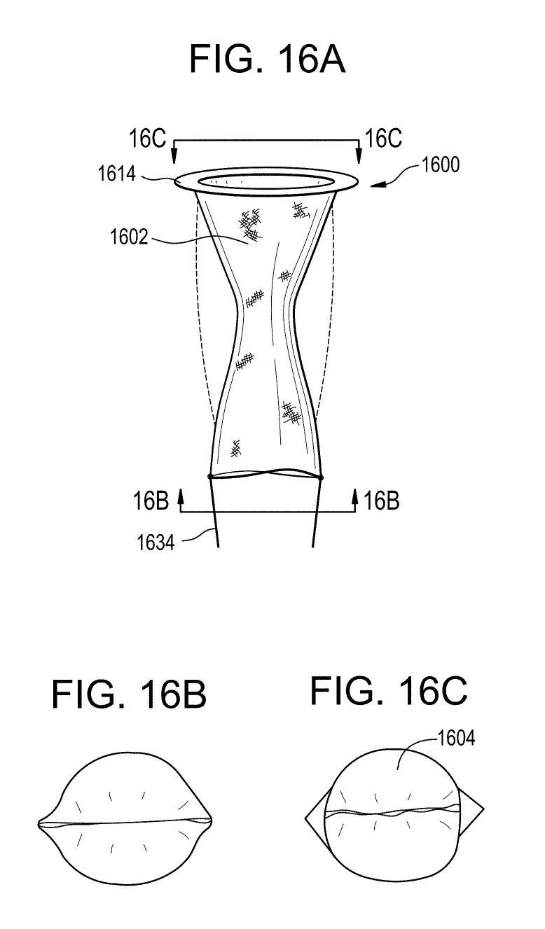

FIG. 16(a)-(c) is a multi-component view of an illustration of an hourglass-shaped, two-tether 1634, cable-type (toroid) top stent 1614 embodiment of the present invention. FIG. 16(a) shows an illustration of an entire device 1600. FIG. 16(b) shows a bottom view along line B-B and shows how the cylinder 1602 collapses to a closed position. FIG. 16(c) shows a top view along line A-A looking down the interior of the channel 1604. FIG. 16 also shows bottom stent/anchor 1626.

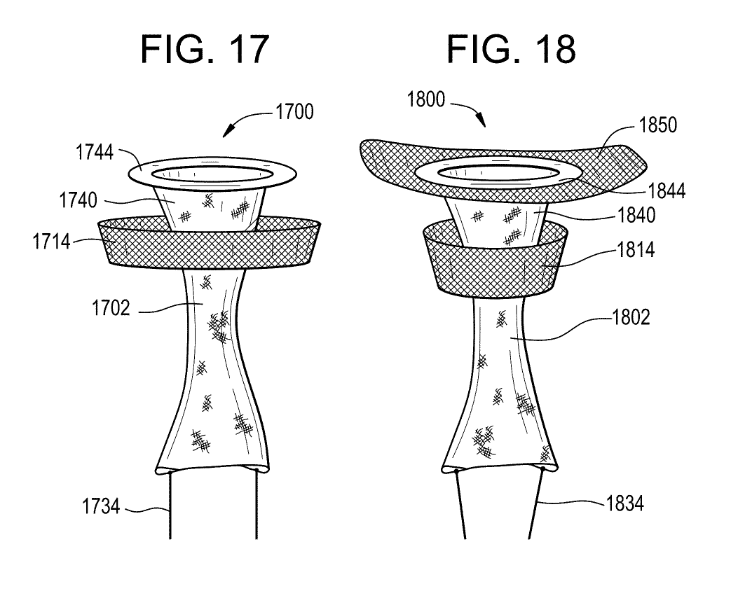

FIG. 17 is an illustration of another embodiment of the present device 1700 and shows a cable-style (toroidal) collar 1744 attached to an hourglass shaped cylinder/conduit 1702 that has a wide-aspect top stent/frame 1714 mounted around the cylinder 1702. FIG. 17 shows a two tether 1734 embodiment and a low-aspect bottom-stent style anchor 1726.

FIG. 18 is an illustration of another embodiment of the present device 1800 and shows a cable-style (toroidal) collar 1844 with a large panel 1850 attached to an hourglass shaped conduit/cylinder that has a narrow-aspect top stent/frame 1814 mounted around the cylinder 1802. FIG. 18 shows a two tether 1834 embodiment and a narrow-aspect bottom-stent style anchor 1826.

FIG. 19 is an illustration of another embodiment of the present device 1900 and shows a cable-style (toroidal) collar 1944 with a large panel 1950 attached to an hourglass shaped conduit 1902 but does not have any top stent mounted around the cylinder/conduit 1902. FIG. 19 shows a two tether 1934 embodiment and a low-aspect bottom-stent style anchor 1926.

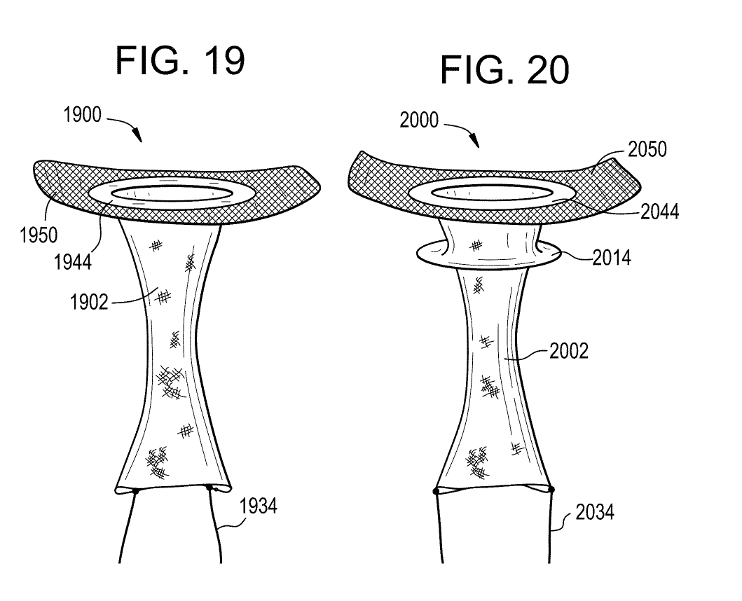

FIG. 20 is an illustration of another embodiment of the present device 2000 and shows a cable-style (toroidal) collar 2044 with a large panel 2050 attached to an hourglass shaped cylinder 2002 and has a covered-frame style top stent 2014 mounted around the cylinder/conduit 2002. FIG. 20 shows a two tether 2034 embodiment and a low-aspect bottom-stent style anchor 2026.

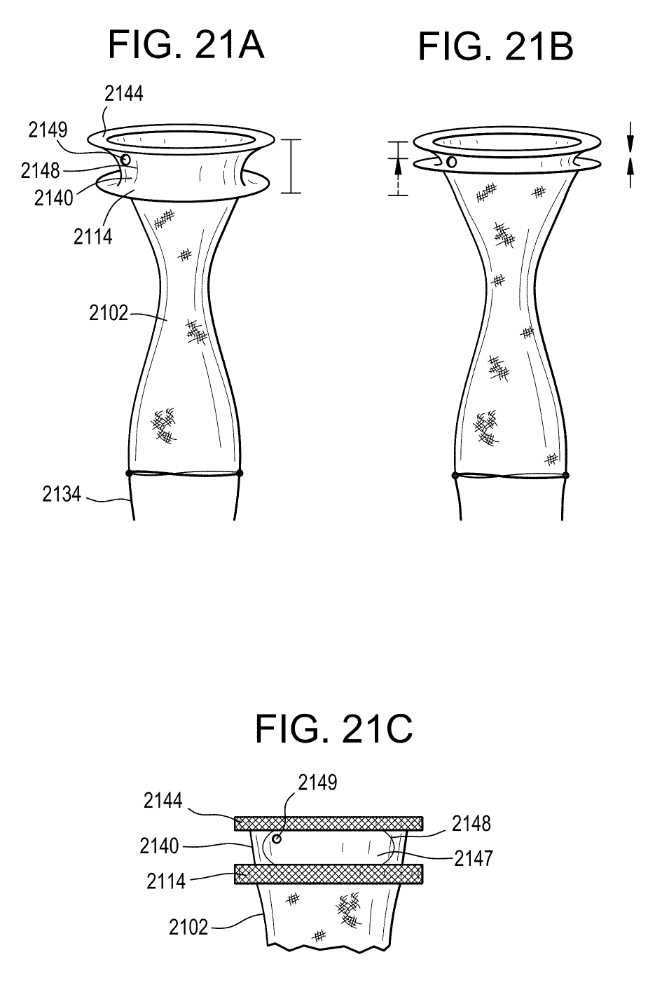

FIGS. 21(a) and 21(b) is an illustration of another embodiment of the present device 2100 and shows a vacuum-mounting feature. FIGS. 21(a) and (b) show a time-sequence of the deflation of a filled compartment. FIGS. 21(a) and (b) show an embodiment whereby a cable-style (toroidal) collar 2144 is attached to an hourglass shaped cylinder 2102 (conduit) that has a covered-frame style top stent 2114 mounted around the cylinder 2102, but where the top stent 2114 has a covered nitinol frame that supports a deflatable ring 2148, wherein the deflatable ring 2148 is comprised of a toroid-shaped sealed compartment 2147 (within cover) having a valve 2149, said sealed compartment 2147 fillable with a biocompatible liquid or gas, wherein upon removal of some or all of the biocompatible liquid or gas, the deflatable ring 2148 works in cooperation with the (non-moving) collar 2144 to compress the top spacer segment 2140 of the cylinder to a reduced height and thereby operate to seal and mount the device within a native annulus. FIGS. 21(a) and (b) shows a two tether 2134 embodiment and a moderator band anchor 2126. FIG. 21(c) shows a cross-sectional view, sans cover.

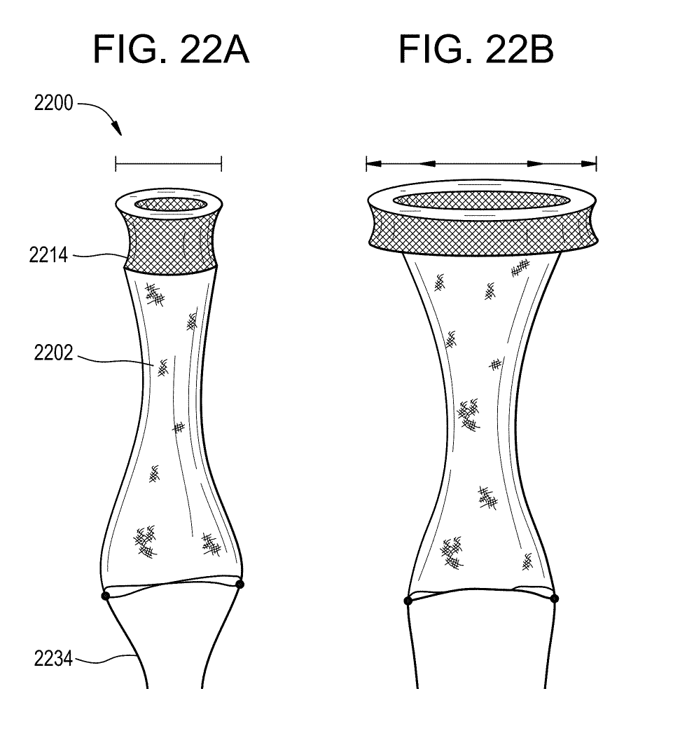

FIGS. 22(a) and 22(b) are illustrations of another embodiment of the present device 2200 and shows in sequence an expansion-mounting feature whereby a compressed top-stent 2214 is attached to an hourglass shaped cylinder 2202 but whereby the top-stent 2214 and the bottom stent 2226 are comprised of a compressed material that is released, or of an inelastic deformable material, and thereby operate to seal and mount the device within a native annulus and native mount-area. FIG. 22 shows a two tether 2234 embodiment and a low-aspect bottom-stent style anchor 2226.

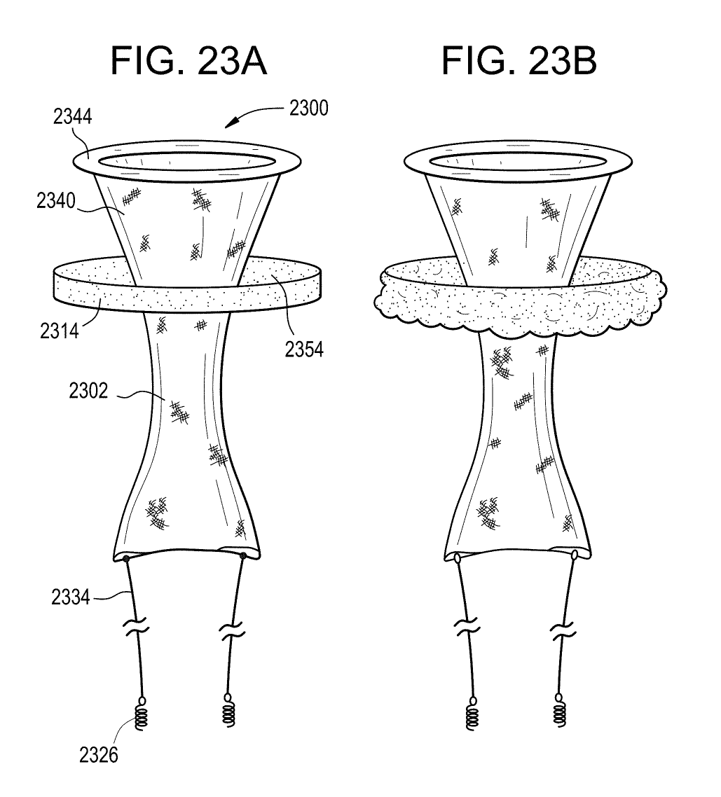

FIGS. 23(a) and 23(b) are illustrations of another embodiment of the present device 2300 and shows in sequence an inflatable (or swellable)-mounting feature whereby a cable-style (toroidal) collar 2344 is attached to an hourglass shaped cylinder 2302 that has an uninflated or undeveloped top-stent 2314 attached to the hourglass shaped cylinder 2302. FIG. 23(b) shows whereby the top-stent 2314 with polymer matrix 2354 absorbs liquid and expands, and thereby operates to seal and mount the device within a native annulus. FIGS. 23(a) and (b) show a two tether 2334 embodiment and, e.g. a tissue anchor(s) 2326.

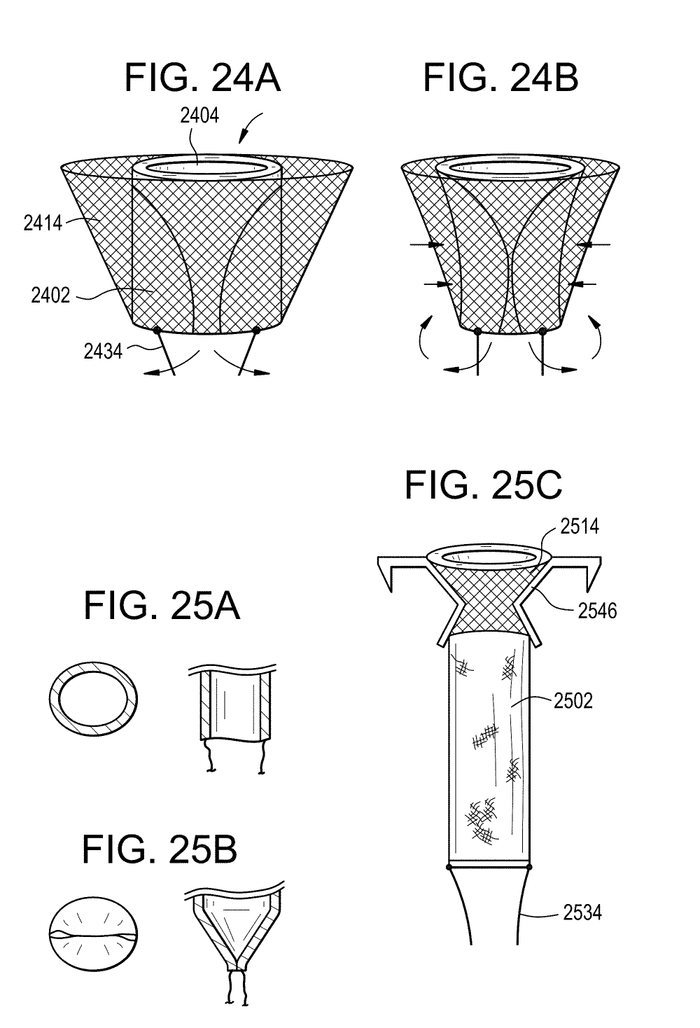

FIGS. 24(a) and 24(b) are illustrations of another embodiment of the present device and show in sequence a thick walled cylinder 2402 being compressed by external pressure and closing the channel 2404. FIGS. 24(a) and (b) show a two tether 2434 embodiment and a low-aspect bottom-stent style anchor 2426. FIG. 24 also shows how frame 2414 can be configured to be approximately the same height of the conduit 2402.

FIG. 25(a)-(c) is an illustration of a multiple components on one embodiment of the present invention. FIG. 25(a) shows a cross-section of an open channel having two-tethers. FIG. 25(b) shows a cross-section of a compressed cylinder and closed channel having two tethers. FIG. 25(c) shows an embodiment of the prosthetic medical device having a top stent 2514 attached to a collapsible cylinder conduit 2502, the top stent 2514 having two contralateral annular anchors 2546, and a two tether 2534 embodiment and a low-aspect bottom-stent style anchor 2526.

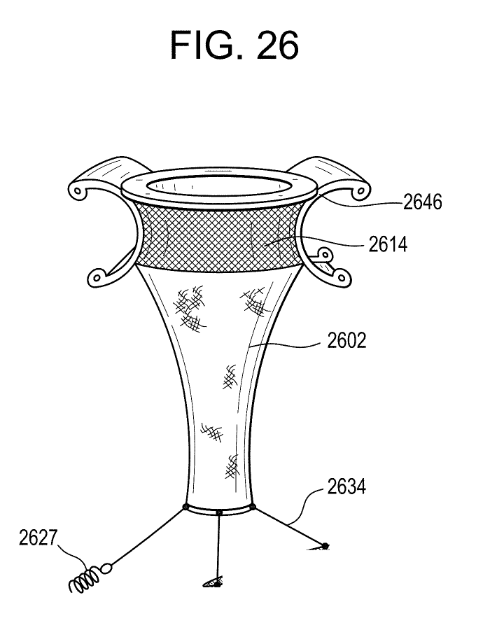

FIG. 26 shows an embodiment of the prosthetic medical device having a top stent 2614 attached to a conic cylinder conduit 2602, the top stent 2614 having two contralateral annular anchors 2646, and a three tether 2634 embodiment with two tethers attached to a low-aspect bottom-stent style anchor 2626, and one tether attached to a tissue anchor 2627.

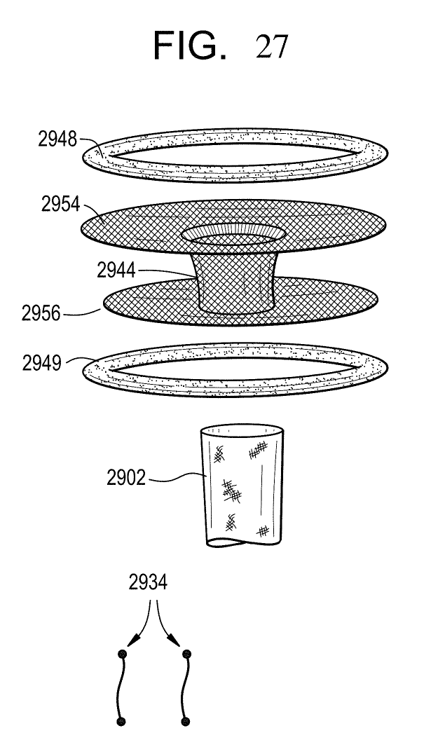

FIG. 27 is an illustration of another embodiment of the present device and shows a central stent hub 2944 with aperture 2904 and having a top (apical) circumferential flange 2954 and a bottom (ventricular) circumferential flange 2956 connected to the hub 2944, with a top toroidal inflatable ring 2948 attached to the top (apical) circumferential flange 2954 and a bottom toroidal inflatable ring 2949 attached to the bottom (ventricular) circumferential flange 2956. FIG. 27 is an exploded view and shows the component parts of one embodiment. Fillable (or filled or compressive matrix) top ring 2948 mounts atop top circumferential flange 2954, and which is in urn connected to central stent hub 2944. Hub 2944 is connected to bottom circumferential flange 2956, and which has bottom ring 2949 disposed on its bottom surface. Pliant tubular conduit 2902 is connected in communication with the central aperture of the hub 2944. Tethers 2934 connect conduit 2902 to bottom anchor/stent 2926.

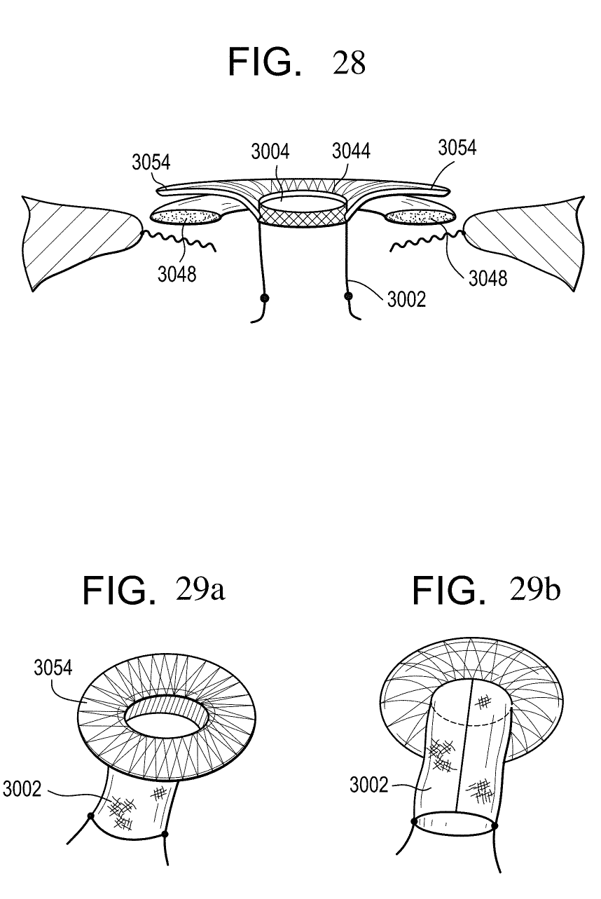

FIG. 28 shows a central stent hub 3044 with aperture 3004 and having a top (apical) circumferential flange 3054 connected to the hub 3044, with a top toroidal inflatable ring 3048 attached to the top (apical) circumferential flange 3054. FIG. 28 is a cross-sectional side view and shows how the native leaflet, indicated by wavy line, sandwiches the ring and forms a seal to prevent regurgitation during systole.

FIG. 29(a) is a perspective top view and shows how the top surface of the flange may be left as open mesh stent material. FIG. 29(b) is a perspective bottom view and shows the native leaflet flattened and compressed by the inflatable ring above it (not seen). FIG. 29(b) also shows pliant tubular channel 3002 is attached to the subannular aperture annulus and leading into the ventricle.

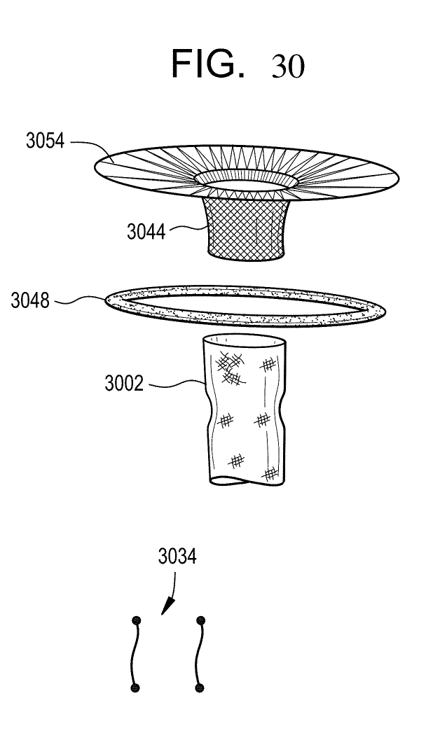

FIG. 30 is an exploded view and shows top flange 3054 connected to central hub 3044. Sealing ring 3048 is mounted on the underside of the flange 3054. Conduit 3002 for a channel with and is in communication with the interior channel of hub 3044. Tethers 3034 connect conduit to bottom anchor 3026.

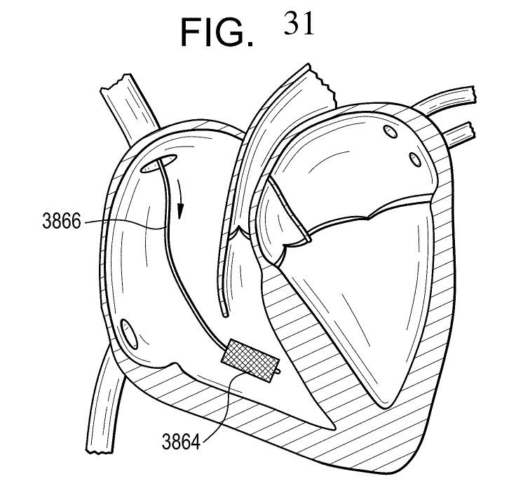

FIG. 31 is an illustration showing a balloon expanding delivery catheter 3866 delivering a compressed, unexpanded heart repair device 3864 to the right ventricle.

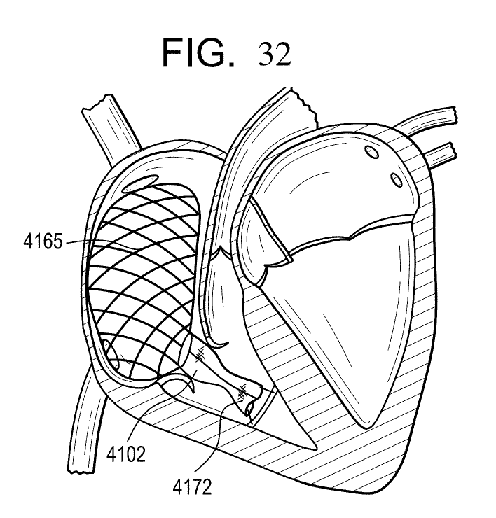

FIG. 32 is an illustration of a passive assist cage device deployed in the right atrium with pliant tubular conduit extending through the tricuspid valve annulus into the right ventricle. In this embodiment, semi-rigid conduit support is shown attached to or within conduit.

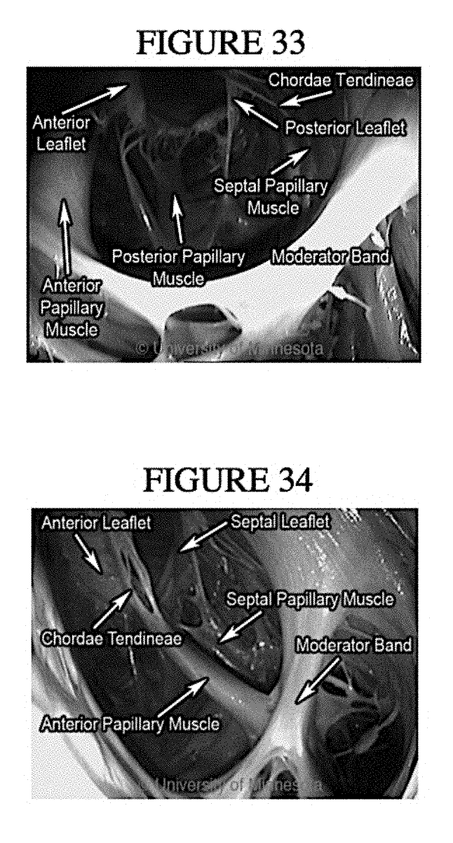

FIG. 33 is a photographic illustration showing an view from the apex towards the tricuspid within the right ventricle and shows the moderator band in the foreground right, anterior papillary muscle left side, anterior leaflet top rear, posterior leaflet along with chordae tendinae top middle-right, and septal papillary muscle right side.

FIG. 34 is a photographic illustration showing a view from the apex-parietal side towards the tricuspid within the right ventricle and shows the moderator band in the foreground right, anterior papillary muscle left side, anterior leaflet top rear, posterior leaflet along with chordae tendinae top middle-right, septal papillary muscle right side, and septal leaflet center rear.

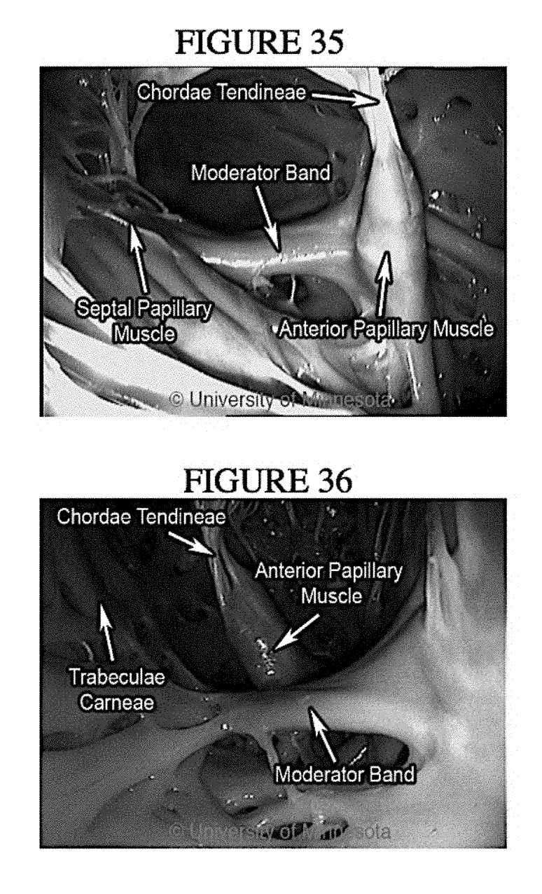

FIG. 35 is a photographic illustration showing a view down the tricuspid aperture towards the right ventricular apex and shows the moderator band in the center, anterior papillary muscle right side, septal papillary muscle left side, and chordae tendinae top right.

FIG. 36 is a photographic illustration showing a downward view across the right ventricle from the septal wall towards the parietal/apex and shows the moderator band in the center, anterior papillary muscle behind, chordae tendinae and trabeculae carneae convolutions left side.

Transcatheter Delivery

During use, the transcatheter delivery apparatus includes a delivery sheath assembly, a handle and an outer stability tube. The delivery sheath assembly defines a lumen, and includes a distal capsule and a proximal shaft. The capsule is configured to compressively contain the heart valve prosthesis. The shaft is coupled to the capsule such that longitudinal movement of the shaft is transferred to the capsule. The handle includes a housing and an actuator mechanism. The housing defines a proximal side and a distal side. The actuator mechanism is maintained by the housing and is coupled to the shaft, with the shaft extending distal the distal side of the housing. Further, the actuator mechanism is configured to selectively move the shaft, and thus the capsule, relative to the housing. The outer stability tube is coupled to the housing and is coaxially received over the shaft such that the shaft is slidable relative to the stability tube. Finally, a distal end of the stability tube terminates proximal the capsule in at least a distal-most arrangement of the delivery sheath assembly. With the above in mind, the actuator mechanism is operable to transition the delivery device from a loaded or delivery state to a deployed state. In the loaded state, the capsule encompasses the implantable device to be deployed, e.g. a moderator band anchor, or a prosthetic heart valve. In the deployed state, the capsule is withdrawn from the implant. In this regard, the shaft slides relative to the stability tube in transitioning from the delivery state to the deployed state. In some embodiments, the delivery device is used in conjunction with an introducer device for delivering the implant into the patient's vasculature, with the stability tube serving to isolate the delivery sheath from the introducer device.

The delivery devices described herein can be modified for delivery of balloon-expandable stented heart valves, within the scope of the present disclosure. Delivery of balloon-expandable stented heart valves can be performed percutaneously using modified versions of the delivery devices of the present disclosure. In general terms, this includes providing the transcatheter delivery assembly akin to those described above, along with a balloon catheter and a guide wire.

To access a bodily lumen (e.g., femoral artery) of the patient, an incision is formed in the patient's skin, and the introducer sheath inserted through the incision and into the desired bodily lumen. The valve fluidly closes the connection with the bodily lumen external the patient. The delivery device is then inserted into the bodily lumen via the introducer device. The introducer sheath has an inner diameter greater than that of the outer stability tube and the capsule, such that the capsule can readily be delivered through the bodily lumen, directed to other branches of the patient's vasculature, and then to the implantation site. In this regard, the introducer valve frictionally contacts the outer stability tube, thereby establishing a low friction hemostasis seal around the outer stability tube. Notably, however, the outer stability tube isolates the delivery sheath assembly and in particular the shaft from the introducer sheath and valve. While the outer stability tube is in physical contact with portions of the introducer device, the delivery sheath assembly does not directly contact the introducer device. Further, the stability tube overtly supports the delivery shaft in traversing the tortuous vasculature, minimizing occurrences of kinks forming in the shaft when moving across the curved portions of the heart.

Anchor Deployment

Anchors are deployed by overwire delivery of an anchor or anchors through a delivery catheter. The catheter may have multiple axial lumens for delivery of a variety of anchoring tools, including anchor setting tools, force application tools, hooks, snaring tools, cutting tools, radio-frequency and radiological visualization tools and markers, and suture/thread manipulation tools. Once the anchor(s) are attached to the moderator band, tensioning tools may be used to adjust the length of tethers that connect to an implanted valve to adjust and secure the implant as necessary for proper functioning. It is also contemplated that anchors may be spring-loaded and may have tether-attachment or tether-capture mechanisms built into the tethering face of the anchor(s). Anchors may also have in-growth material, such as polyester fibers, to promote in-growth of the anchors int the myocardium.

### LIST OF REFERENCES NUMBERS

100 prosthetic medical device tricuspid
102 elongated flexible cylinder (pliant tubular conduit)
104 cylinder channel/conduit lumen
106 top end
108 bottom end
109 actuator handle of delivery device
110 internal surface
111 flexible percutaneous delivery catheter, external, proximal
112 external surface
113 cylinder/conduit mid-segment side wall
114 top stent/resilient annular or subannular frame
115 flexible percutaneous delivery catheter, internal RA, distal
116 top stent channel
117 catheter tool
118 top stent side wall
119 moderator band cincture
120 top stent top aperture
121 moderator band
122 tether loop/mount
123 moderator band collar with anchor loop
124 top stent cover
126 moderator band suture or coil anchor
128 linkage, tether-to-collar
130 piercing tissue anchor
131 helical member
132 release or feed mechanism
133 cavity
134 2-5 tethers
135 anchor sheath
136 conic cylinder
137 anchor manipulation tool
138 top edge of cylinder top end
140 screw-type tissue anchor
141 screw member
142 release or feeder mechanism
144 collar
145 anchor sheath
147 anchor manipulation tool
160 two-component tissue anchor
161 accordion/multi-fold suture or Nitinol anchor
162 anchor tool
163 delivery catheter
165 far side Tbar tether anchor
165 near side Tbar tether anchor
166 spanning tether section
170 two component tissue anchor
171 accordion/multi-fold suture or Nitinol anchor
174 far side Tbar tether anchor
175 near side Tbar tether anchor
176 spanning tether section
180 clamp/clip anchor
181 anchor tool
182 first control arm
183 second control arm
184 first bracket member
185 second bracket member
186 third control arm
187 bracket locking sleeve
189 spring
190 barbed anchor
191 anchor tool
192 barb
193 shaft
194 delivery catheter
195 sheath
196 penetrating point
1400 prosthetic medical device
1402 elongated flexible cylinder (pliant tubular conduit)
1414 sub-annular stent/frame
1434 2-5 tethers
1500 prosthetic medical device
1502 elongated flexible cylinder (pliant tubular conduit)
1514 sub-annular stent/frame
1534 2-5 tethers
1600 prosthetic medical device
1602 elongated flexible cylinder (pliant tubular conduit)
1604 channel
1614 sub-annular stent/frame
1634 2-5 tethers
1700 prosthetic medical device
1702 elongated flexible cylinder (pliant tubular conduit)
1714 sub-annular stent/frame
1734 2-5 tethers
1744 toroid collar
1800 prosthetic medical device
1802 elongated flexible cylinder (pliant tubular conduit)
1814 sub-annular stent/frame
1834 2-5 tethers
1844 toroid collar
1850 large panel
1900 prosthetic medical device
1902 elongated flexible cylinder (pliant tubular conduit)
1914 sub-annular stent/frame
1934 2-5 tethers
1944 toroidal collar
1950 large panel
2000 prosthetic medical device
2002 elongated flexible cylinder (pliant tubular conduit)
2014 sub-annular stent/frame
2034 2-5 tethers
2044 toroidal collar
2050 large panel
2100 prosthetic medical device
2102 elongated flexible cylinder (pliant tubular conduit)
2114 sub-annular stent/frame
2134 2-5 tethers
2140 top spacer
2144 toroidal collar
2147 compartment
2148 deflatable ring
2149 valve
2150 large panel
2200 prosthetic medical device
2202 elongated flexible cylinder (pliant tubular conduit)
2214 sub-annular stent/frame
2234 2-5 tethers
2244 toroidal collar
2250 large panel
2300 prosthetic medical device
2302 elongated flexible cylinder (pliant tubular conduit)
2314 sub-annular stent/frame
2334 2-5 tethers
2344 toroidal collar
2354 polymer matrix
2400 prosthetic medical device
2402 elongated flexible cylinder (pliant tubular conduit)
2404 channel
2414 sub-annular stent/frame
2434 2-5 tethers
2500 prosthetic medical device
2502 elongated flexible cylinder (pliant tubular conduit)
2504 channel
2514 sub-annular stent/frame
2534 2-5 tethers
2546 contralateral annular anchor
2600 prosthetic medical device
2602 elongated flexible cylinder (pliant tubular conduit)
2604 channel
2614 sub-annular stent/frame
2627 tissue anchor
2634 2-5 tethers
2646 contralateral annular anchor
2902 pliant tubular conduit
2904 aperture
2934 tethers
2944 central stent hub
2948 top toroidal inflatable ring
2949 bottom toroidal inflatable ring
2954 top (apical) circumferential flange
2956 bottom (ventricular) circumferential flange
3002 pliant tubular conduit
3004 aperture
3034 tethers
3044 central stent hub
3048 top toroidal inflatable ring
3054 top (apical) circumferential flange
3864 compressed, unexpanded passive assist cage device
3866 balloon expanding delivery catheter
4102 conduit
4165 uncompressed, expanded passive assist cage device
4172 Semi-rigid conduit support
4134 two or more tethers
331 moderator band
332 posterior pappilary muscle
333 anterior papillary muscle
334 anterior leaflet
335 posterior leaflet
336 chordae tendinae
337 septal papillary muscle
338 septal leaflet
339 trabeculae carneae

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

Having described embodiments for the invention herein, it is noted that modifications and variations can be made by persons skilled in the art in light of the above teachings. It is therefore to be understood that changes may be made in the particular embodiments of the invention disclosed which are within the scope and spirit of the invention as defined by the appended claims. Having thus described the invention with the details and particularity required by the patent laws, what is claimed and desired protected by Letters Patent is set forth in the appended claims.

1. A medical implant, comprising:
a tether platform for attaching one or more tethers to the septomarginal trabecula (moderator band) of the right ventricle for securing and positioning a heart valve repair device, said tether platform sized for transcatheter delivery and deployment within the right ventricle;
said tether platform having a moderator band tissue anchor and an attachment head;
said moderator band tissue anchor selected from a belt anchor, a piercing anchor, and a combination thereof;
said belt anchor comprising a strip or loop of material configured to encircle the septomarginal trabecula, said belt anchor having a structure selected from a mesh, a fabric, a braid, a coil, windings, and combinations thereof, said belt anchor constructed of material selected from a biocompatible tissue, a Nitinol wire, a Nitinol mesh, a polymer, and combinations thereof; said belt anchor connected to an attachment head comprising one or more tether stays, said tether stays selected from a loop, a cleat, an anchor, a fastener, a linkage, and combinations thereof;
said piercing anchor comprising a structurally rigid material configured to penetrate tissue of the septomarginal trabecula, said piercing anchor having a structure selected from a helical tissue anchor, a screw-type tissue anchor, an accordion-style multi-fold suture anchor, a multi-fold Nitinol anchor, a T-bar tissue anchor, a clip tissue anchor having two or more rigid arms, a barbed anchor, and combinations thereof; said piercing anchor connected to an attachment head comprising one or more tether stays, said tether stays selected from a loop, a cleat, an anchor, a fastener, a linkage, and combinations thereof;
said attachment head connected to a tether of a heart valve repair device, said tether ranging from about 2.5 cm to 6.6 cm in length;
said moderator band tissue anchor comprising a piercing anchor for piercing the moderator band or a wrapping anchor for grasping or encircling the moderator band, where the piercing anchor is sized to pierce tissue ranging from about 2.04 mm to 7.05 mm in thickness, where the wrapping anchor is sized to encircle a circumference ranging from about 6.0 mm to about 22.0 mm; and
wherein the heart valve repair device comprises a replacement valve or a bypass valve selected from the group consisting of: (i) an expandable shape memory replacement valve deployed in the tricuspid annulus, or (ii) a reciprocating conduit valve, where said reciprocating conduit valve is a pliant tubular conduit mounted on a resilient annular frame and tethered within the right ventricle of the heart, wherein the pliant tubular conduit is a reciprocating mechanical member that is compressed by pressurized working fluid within the ventricle during systole.

2. The medical implant of claim 1, wherein the piercing anchor is selected from the group consisting of: (i) a helical tissue anchor, (ii) a screw-type tissue anchor, (iii) a near-side T-bar tether anchor connected by a spanning tether section to a far-side T-bar tether anchor, (iv) far-side T-bar tether anchor connected by a bridge tether section to a pair of near-side tether anchors, where the near-side tether anchors are made from shape memory alloy and deform from a tubular sheath shape around the tether to a flattened, compressed disc shape by pulling the tether to draw the distal end of the sheath to the proximal end of the sheath; and (v) an arrow-shape tissue anchor that is compressed with a sheath, wherein the sheath is withdrawn to allow the memory-shape spring loaded metal arrow to open and laterally expand a pair of arrow points after the pointed sheath has penetrated the tissue to be anchored.

3. The medical implant of claim 1, wherein the belt anchor is selected from the group consisting of: (i) a bracket-type anchor, (ii) one or more loops of material as anchor, (iii) a compression band type of anchor, (iv) a wire mesh anchor, and (v) a clamp type anchor.

4. The medical implant of claim 1, wherein the tissue anchor is connected to a tether mounting ring or tether mounting hook.

5. The medical implant of claim 1, wherein the moderator band tissue anchor comprises between 2-individual tissue anchors, each tissue anchor having a mounting ring or hook attached thereto.

6. A method for providing an anchor mount for securing and positioning a heart valve repair device within the right ventricle, comprising the steps:
(i) loading tether platform within the lumen of a transcatheter delivery system and percutaneously accessing a right side of a heart;
(ii) anchoring the tether platform to the septomarginal trabecula (moderator band) of the right ventricle for securing and positioning a heart valve repair device, said tether platform sized for transcatheter delivery and deployment within the right ventricle, said tether platform having an attachment head and a moderator band tissue anchor;
said attachment head positioned within the right ventricle for connecting a tether of a heart valve repair device, said tether ranging from about 2.5 cm to 6.6 cm in length;
said moderator band tissue anchor comprising a piercing anchor for piercing the moderator band or a wrapping anchor for grasping or encircling the moderator band, where the piercing anchor is sized to pierce tissue ranging from about 2.04 mm to 7.05 mm in thickness; and
wherein the heart valve repair device comprises a replacement valve or a bypass valve selected from the group consisting of: (i) an expandable shape memory replacement valve deployed in the tricuspid annulus, or (ii) a reciprocating conduit valve, where said reciprocating conduit valve is a pliant tubular conduit mounted on a resilient annular frame and tethered within the right ventricle of the heart, wherein the pliant tubular conduit is a reciprocating mechanical member that is compressed by pressurized working fluid within the ventricle during systole.
